(19) United States
(12) Patent Application Publication
(10) Pub. No.: US 2009/0325923 A1
(43) Pub. Date: Dec. 31, 2009

(54) NEW METHOD FOR THE TREATMENT OF INFLAMMATORY DISEASES

(75) Inventors: Oberdan Leo, Wezembeek-Oppem (BE); Thibaut De Smedt, Prevessin-Moens (FR); Frederic Van Gool, Kraainem (BE); Mara Galli, Charleroi (BE)

Correspondence Address: Pepper Hamilton LLP, 400 Berwyn Park, 899 Cassatt Road, Berwyn, PA, 19312-1183, US

(73) Assignee: TOPOTARGET SWITZERLAND SA, Lausanne (CH)

(21) Appl. No.: 12/439,276

(22) PCT Filed: Sep. 3, 2007
(86) PCT No.: PCT/EP2007/059193

§ 371 Date: Jul. 2, 2009

(30) Foreign Application Priority Data
Sep. 1, 2006 (IB) PCT/IB2006/002402

(51) Int. Cl. A61K 31/4427; A61K 31/4545; C07D 401/12

(52) U.S. Cl. 514/210.2; 514/318; 546/194

(57) ABSTRACT

The present invention relates to the use of an inhibitor of the formation of nicotinamide adenyl dinucleotide for the preparation of a medicament used in the treatment of inflammatory diseases such as rheumatoid arthritis and endotoxemia.

The present invention relates to the use of an inhibitor of the formation of nicotinamide adenyl dinucleotide for the preparation of a medicament used in the treatment of inflammatory diseases. The invention relates also to a process to manufacture a medicament for treating inflammatory diseases and finally to a pharmaceutical kit comprising such inhibitor.

The preparation of a class of compounds, comprising several subclasses, which act as inhibitors of the formation of nicotinamide adenyl nucleotide, and their use thereof as anti-tumour agents, is already described in the patent applications WO0050399, WO97/48695, WO97/48397, WO99/31063, WO9931060, WO9931087, WO9931064, WO00/50399, and WO0380054. Especially useful compounds are described in the PCT application WO9748696.

One of these inhibitors, (E)-N-[4-(1-benzoylpiperidin-4-yl)butyl]-3-(pyridine-3-yl)-acrylamide also known as APO866, FK866, WK175, or WK22.175 and hereinafter referred to as FK866 [International Non-proprietary Name], is especially described in the literature as an anticancer agent.

FK866 may be used for treatment of diseases implicating deregulated apoptosis such as cancer. It has been demonstrated in the prior art that FK866 interferes with nicotinamide adenyl dinucleotide (also known and hereinafter referred to as NAD) biosynthesis and induces apoptotic cell death without any DNA damaging effects.

Antiangiogenic and antitumoral efficacy of FK866 are described in many publications.

The publication “FK866, a high specific non-competitive inhibitor of nicotinamide phosphoribosyltransferase, represents a novel mechanism for induction of tumor cell apoptosis”, M. Hasmann et al., Cancer Research 63, 7436-7442, Nov. 1, 2003 describes more generally FK866 as the first high potent and specific inhibitor of nicotinamide phosphoribosyltransferase and its characteristics as antitumor compound.

For example, its efficacy as antitumor agent for the treatment of leukaemia is described in “WK175, a novel antitumor agent, decreases the intracellular nicotinamide adenine dinucleotide concentration and induces the apoptotic cascade in human leukaemia cells”, K. Wosikowski et al., Cancer Research 62, 1057-1062, Feb. 15, 2002.

Its efficacy as antitumor agent for the treatment of renal carcinoma is demonstrated in “antiangiogenic potency of FK866/K22.175, a new inhibitor of intracellular NAD biosyntheses, in murine renal cell carcinoma”, J. Dreves et al., Anticancer Research 23: 4853-4858 (2003).

EP 1 348 434 describes the use of pyridyl amides including FK866 as inhibitors of angiogenesis. According to this document a number of diseases are characterized by unregulated angiogenesis such as inflammatory disorder, proliferative retinopathies, rheumatoid arthritis, macular degeneration, preneoplastic lesions, benign prostatic hyperplasia, venous neointimal hyperplasia and psoriasis. However, EP 1 348 434 only describes the effect of FK 866 on angiogenesis in a murine renal cell carcinoma. Moreover, it is probable that for most inflammatory diseases including rheumatoid arthritis, angiogenesis is a consequence, rather than a cause, of inflammation.

Considering the above-mentioned completely different known medical indications of known inhibitors to the formation of nicotinamide adenyl dinucleotide, the activity of the compounds used according to the invention with advantageous therapeutic properties for inflammatory diseases was completely surprising for the person skilled in the art.

The notion of inflammatory diseases delineates a heterogeneous group of pathologies that involve innate or adaptive immune system components and characterized by chronic inflammation in the absence of infection or seemingly unprovoked. Examples of these diseases are hereditary periodic fevers, Muckle-Wells syndrome, familial mediterranean fever, familial cold-induced autoinflammatory syndrome, rheumatoid arthritis, systemic onset juvenile idiopathic arthritis, osteoarthritis, Crohn's disease, multiple sclerosis, the metabolic disorders gout and pseudogout, atherosclerosis, Alzheimer disease and Parkinson disease.

Tumor necrosis factor-α (TNF-α), interleukin-1 (IL-1), and interleukin-6 (IL-6) are cytokines produced by cells of the innate immune system upon microbial activation, and are important mediators of both local and systemic inflammation. In many instances, the secretion of these cytokines is deregulated in inflammatory diseases leading to chronic inflammation.

Inhibition of TNF-α, IL-1, and IL-6 production is beneficial in several inflammatory diseases, and numerous efforts have been devoted in the design of novel therapies aimed at blocking the production and/or the biological effects of these important pro-inflammatory cytokines.

In the present invention, unexpected anti-inflammatory properties of the inhibition of nicotinamide adenyl dinucleotide have been identified. By inhibiting nicotinamide adenyl dinucleotide biosynthesis, it was surprisingly found that TNF-α, IL-1 and IL-6 secretion are inhibited.

In the present invention, it is demonstrated that optimal proinflammatory cytokine levels, including TNF-α, IL-1 and IL-6, require adequate nicotinamide adenyl dinucleotide intracellular concentration.

In view of this art, the finding that inhibitors of the formation of nicotinamide adenyl dinucleotide have activities which make them particularly suitable in an excellent manner for the therapy of inflammatory diseases was completely unexpected.

The present invention establishes a functional link between metabolism and inflammation, and demonstrates a potential important role for NAD-dependent enzymes in the regulation of proinflammatory cytokine synthesis, including TNF-α, IL-1 and IL-6.

Hence, in a first embodiment, the present invention relates to the use of an inhibitor of the formation of nicotinamide adenyl dinucleotide for the preparation of a medicament used in the treatment of inflammatory diseases.

In a second embodiment, the present invention relates to a process to manufacture a medicament for treating inflammatory diseases.

In a third embodiment, the present invention also concerns a method of treating inflammatory diseases comprising administering to a subject an effective amount of an inhibitor of the formation of nicotinamide adenyl dinucleotide.

Furthermore, the present invention concerns also a pharmaceutical kit comprising at least an effective amount of an inhibitor of the formation of nicotinamide adenyl dinucleotide together with instructions for use in the treatment of inflammatory diseases.

The term “inhibitor” refers to a substrate molecule that blocks a particular biologic activity.

The expression “competitive inhibitor” refers to a substrate molecule which directly binds to the same active site as the normal enzyme substrate, without undergoing a reaction.

The expression “non-competitive inhibitor” as used herein defines a substrate molecule which always binds to the enzyme at a site other than the enzyme's active site. The binding affects the activity of the enzyme because the presence of the inhibitor causes a change in the structure and shape of the enzyme but it doesn't change the apparent binding affinity of the normal enzyme substrate.

The term “inflammatory diseases” refers to diseases that are characterized by activation of the immune system to abnormal levels that lead to disease.

The terms “rheumatoid arthritis” refer to chronic, inflammatory autoimmune disorder that causes inflammation of the joints.

The expression “effective amount” generally refers to the quantity for which the active substance has therapeutical effects. In the present case the active substance is the inhibitor of the formation of nicotinamide adenyl dinucleotide.

“Nicotinamide phosphoribosyltransferase” also named NMPRT, NMPRTase or NAmPRTase, (International nomenclature: E.C. 2.4.2.12) is a key enzyme in nicotinamide adenyl dinucleotide (NAD) biosynthesis from the natural precursor nicotinamide.

“Nicotinic acid” is a precursor of NAD.

In the present invention, both terms “TNF” and “TNF-α” are used to designate the cytokine named “Tumor necrosis factor-α.”.

When used as a therapeutic the inhibitor of the formation of nicotinamide adenyl dinucleotide described herein are preferably administered with a physiologically acceptable carrier. A physiologically acceptable carrier is a formulation to which the compound can be added to dissolve it or otherwise facilitate its administration. Example of a physiologically acceptable carrier includes propylene glycol. An important factor in choosing an appropriate physiologically acceptable carrier is choosing a carrier in which the compound remains active or the combination of the carrier and the compound produces an active compound.

Benefits of the present invention include oral administration of an optimal amount of a NAD biosynthesis inhibitor.

Based on these results, the present invention has important implications for the design of novel treatment strategies for patients with inflammatory diseases.

Thus, a first aspect of the present invention concerns the use of an inhibitor of the formation of nicotinamide adenyl dinucleotide for the preparation of a medicament used in the treatment of inflammatory diseases.

According to the present invention, the inhibitor is preferably a competitive or noncompetitive inhibitor of the enzyme nicotinamide phosphoribosyltransferase. According to the present invention, the inhibitor is preferably a compound of formula (I)

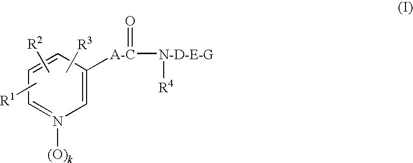

wherein
R¹ is selected from the group consisting of hydrogen, halogen, cyano, C₁-C₆-alkyl, trifluoromethyl, C₃-C₈-cycloalkyl, C₁-C₄-hydroxyalkyl, hydroxy, C₁-C₄-alkoxy, benzyloxy, C₁-C₄-alkanoyloxy, C₁-C₄-alkylthio, C₂-C₅-alkoxycarbonyl, aminocarbonyl, C₃-C₉-dialkylaminocarbonyl, carboxy, phenyl, phenoxy, pyridyloxy, and NR⁵R⁶, wherein
R⁵ and
R⁶ are selected independently from each other from hydrogen and C₁-C₆-alkyl,
R² is selected from hydrogen, halogen, C₁-C₆-alkyl, trifluoromethyl and hydroxy, wherein
R¹ and R², in the case they are adjacent, optionally form a bridge which is selected from the group of bridge members —(CH₂)₄— and —(CH═CH)₂— and —CH₂O—CR⁷R⁸—O—, wherein
R⁷ and
R⁸ are independent from each other, hydrogen or C₁-C₆-alkyl,
R³ is selected from hydrogen, halogen and C₁-C₆-alkyl,
R⁴ is selected from hydrogen, C₁-C₆-alkyl, C₃-C₆-alkenyl, hydroxy, C₁-C₆-alkoxy and benzyloxy,
k is 0 or 1,
A is selected from
   C₂-C₆-alkenylene, which is optionally substituted one to three-fold by C₁-C₃-alkyl, hydroxy, fluorine, cyano, or phenyl,
   C₄-C₆-alkadienylene, which is optionally substituted once or twice by C₁-C₃-alkyl, fluorine, cyano, or phenyl,
   1,3,5-hexatrienylene, which is optionally substituted by C₁-C₃-alkyl, fluorine, or cyano, and
   ethinylene,
D is selected from
   C₁-C₁₀-alkylene, optionally substituted once or twice by C₁-C₃-alkyl or hydroxy,
   C₂-C₁₀-alkenylene, optionally substituted once or twice by C₁-C₃-alkyl or hydroxy, wherein the double bond optionally is to ring E.
   C₃-C₁₀-alkinylene, optionally substituted once or twice by C₁-C₃-alkyl or hydroxy, and
   the group consisting of C₁-C₁₀-alkylene, C₂-C₁₀-alkenylene and C₃-C₁₀-alkinylene, in which one to three methylene units are isosterically replaced by O, S, NR⁹, CO, SO or SO₂, wherein
   R⁹ is selected from hydrogen, C₁-C₃-alkyl, C₁-C₆-acyl and methanesulfonyl,
E is selected from

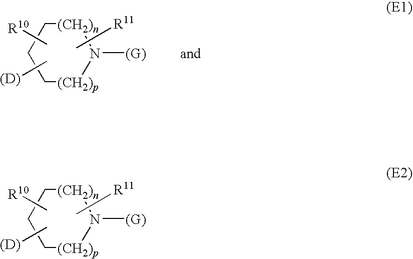

wherein the heterocyclic ring optionally has a double bond and
n and p are, independent of each other, 0, 1, 2 or 3, with the proviso that n+p≦4,
q is 1 or 2,
R¹⁰ is selected from hydrogen, C₁-C₃-alkyl, hydroxy, and hydroxymethyl, carboxy and C₂-C₇-alkoxycarbonyl,
R¹¹ is hydrogen or an oxo group adjacent to the nitrogen atom,
G is selected from hydrogen, G1, G2, G3, G4 and G5, wherein
G1 represents the residue

—(CH₂)ᵣ—(CR¹³R¹⁴)ₛ—R¹² (G1)

wherein
r is 0, 1 or 2 and
s is 0 or 1,
R¹² is selected from hydrogen, C₁-C₆-alkyl, C₃-C₆-alkenyl, C₃-C₆-alkinyl, C₃-C₈-cycloalkyl, benzyl, phenyl,
   the group consisting of monocyclic aromatic five- and six-membered heterocycles, which contain one to three hetero-atoms selected from N, S and O and are either bound directly or over a methylene group,
   the group consisting of anellated bi- and tricyclic aromatic or partially hydrogenated carbocyclic ring systems with 8 to 16 ring atoms and at least one aromatic ring, wherein the bond occurs either over an aromatic or a hydrogenated ring and either directly or over a methylene group, and
   the group consisting of anellated bi- and tricyclic aromatic or partially hydrogenated heterocyclic ring systems with 8 to 16 ring atoms and at least one aromatic ring, wherein one to three ring atoms are selected from N, S and O and the bond occurs either over an aromatic or a hydrogenated ring, and either directly or over a methylene group,
R¹³ has the same meaning as R¹², but is selected independently thereof,
R¹⁴ is selected from hydrogen, hydroxy, methyl, benzyl, phenyl,
   the group consisting of monocyclic aromatic five- and six-membered heterocycles, which contain one to three hetero-atoms selected from N, S and O and are bound either directly or over a methylene group,
   the group consisting of anellated bi- and tricyclic aromatic or partially hydrogenated carbocyclic ring systems with 8 to 16 ring atoms and at least one aromatic ring, wherein the bond occurs either over an aromatic or a hydrogenated ring and either directly or over a methylene group, and
   the group consisting of anellated bi- and tricyclic aromatic or partially hydrogenated heterocyclic ring systems with 8 to 16 ring atoms and at least one aromatic ring, wherein one to three ring atoms are selected from N, S and O and the bond occurs either over an aromatic or a hydrogenated ring and either directly or over a methylene group,
G2 is selected from the residues

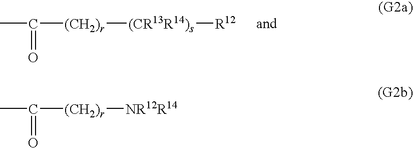

wherein the substituents R¹² and R¹⁴ have the above meaning, or the group

—NR¹²R¹⁴
is a nitrogen-containing heterocycle bound over the nitrogen atom, the nitrogen-containing heterocycle being selected from
the group consisting of saturated and unsaturated monocyclic, four- to eight-membered heterocycles, which, aside from the essential nitrogen atom, optionally contain one or two further hetero-atoms selected from N, S and O, and
the group consisting of saturated and unsaturated bi- or tricyclic, anellated or bridged heterocycles with 8 to 16 ring atoms, which, aside from the essential nitrogen atom, optionally contain one or two further hetero-atoms selected from N, S and O,
G3 is the residue

—SO₂—(CH₂)ᵣR¹² (G3)
G4 is the residue

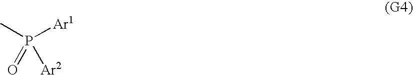

wherein
Ar¹ and
Ar² are selected independently of each other from phenyl, pyridyl and naphthyl,
G5 is the residue

—COR¹⁵ (G5)

wherein
R¹⁵ is selected from trifluoromethyl, C₁-C₆-alkoxy, C₃-C₆-alkenyloxy and benzyloxy,
   wherein aromatic ring systems in the substituents R¹, R², R⁴, R¹², R¹³, R¹⁴, R¹⁵, Ar¹ and Ar² and in the ring system —NR¹²R¹⁴ optionally carry independently of each other one to three substituents which are independently selected from the group consisting of halogen, cyano, C₁-C₆-alkyl, trifluoromethyl, C₃-C₈-cycloalkyl, phenyl, benzyl, hydroxy, C₁-C₆-alkoxy, which is optionally entirely or partially substituted by fluorine, benzyloxy, phenoxy, mercapto, C₁-C₆-alkylthio, carboxy, C₁-C₆-alkoxycarbonyl, benzyloxycarbonyl, nitro, amino, mono-C₁-C₆-alkylamino, and di-(C₁-C₆-alkyl)-amino, wherein two adjacent groups of the aromatic ring or ring system optionally form an additional ring over a methylenedioxy bridge,
   tautomeres in the case of substitution of the heterocycle or in an anellated ring system by free hydroxy, mercapto and/or amino groups,
   stereoisomers and/or mixtures thereof and pharmacologically acceptable acid addition salts with the exception of (E)-3-(3-pyridyl)-N-[2-(1-benzylpiperidin-4-yl)ethyl]-2-propenamide hydrochloride.
   In a preferred embodiment the inhibitor is a compound of formula (I); wherein:
R¹ is selected from hydrogen, halogen, cyano, methyl, trifluoromethyl, hydroxy, C₁-C₄-alkoxy, ethylthio, methoxycarbonyl, tert-butoxycarbonyl, aminocarbonyl, carboxy, and phenoxy,
R² is selected from hydrogen, halogen, trifluoromethyl and hydroxy,
R³ is hydrogen or halogen,
R⁴ is selected from hydrogen, C₁-C₃-alkyl, hydroxy and C₁-C₃-alkoxy,
k is 0 or 1,
A is selected from C₂-C₆-alkenylene, optionally substituted once or twice by C₁-C₃-alkyl, hydroxy or fluorine,
   C₄-C₆-alkadienylene, optionally substituted by C₁-C₃-alkyl or by 1 or 2 fluorine atoms, and 1,3,5-hexatrienylene, optionally substituted by fluorine,
D is selected from C₁-C₈-alkylene, optionally substituted once or twice by methyl or hydroxyl,
   C₂-C₈-alkenylene, optionally substituted once or twice by methyl or hydroxy, wherein the double bond optionally is to ring E,
   C₃-C₈-alkinylene optionally substituted once or twice by methyl or hydroxy, and
   the group consisting of C₁-C₈-alkylene, C₂-C₈-alkenylene and C₃-C₈-alkinylene in which one to three methylene units are isosterically replaced by O, S, NH, N(CH₃), N(COCH₃), N(SO₂CH₃) CO, SO or SO₂,
E is selected from

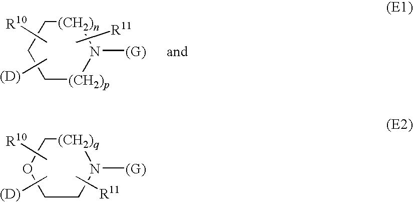

wherein the heterocyclic ring optionally has a double bond and
n and
p are, independent of each other, 0, 1, 2 or 3, with the proviso that n+p≦3,
q is 1 or 2,
R¹⁰ is selected from hydrogen, C₁-C₃-alkyl, hydroxy, and hydroxymethyl,
R¹¹ is hydrogen or an oxo group which is adjacent to the nitrogen atom,
G is selected from hydrogen,
   G1, G2, G3, G4 and G5, wherein
G1 represents the residue

—(CH₂)ᵣ—(CR¹³R¹⁴)ₛ—R¹² (G1)

wherein
r is 0, 1 or 2 and
s is 0 or 1,
R¹² is selected from hydrogen, C₁-C₆-alkyl, C₃-C₈-cycloalkyl, benzyl, phenyl, the group consisting of benzocyclobutyl, indanyl, indenyl, oxoindanyl, naphthyl, dihydronaphthyl, tetrahydronaphthyl, oxotetrahydronaphthyl, biphenylenyl, fluorenyl, oxofluorenyl, anthryl, dihydroanthryl, oxodihydroanthryl, dioxodihydroanthryl, phenanthryl, dihydrophenanthryl, oxodihydrophenanthryl, dibenzocycloheptenyl, oxodibenzocycloheptenyl, dihydrodibenzocycloheptenyl, oxodihydrodibenzocycloheptenyl, dihydrodibenzocyclooctenyl, tetrahydrodibenzocyclooctenyl and oxotetrahydrodibenzocyclooctenyl, bound directly or over a methylene group,
   and
   the group consisting of furyl, thienyl, pyrrolyl, oxazolyl, isoxazolyl, thiazolyl, isothiazolyl, pyrazolyl, imidazolyl, oxadiazolyl, thiadiazolyl, triazolyl, pyridyl, pyrazinyl, pyridazinyl, pyrimidinyl, triazinyl, imidazothiazolyl, benzofuryl, dihydrobenzofuryl, benzothienyl, dihydrobenzothienyl, indolyl, indolinyl, oxoindolinyl, dioxoindolinyl, benzoxazolyl, oxobenzoxazolinyl, benzisoxazolyl, oxobenzisoxazolinyl, benzothiazolyl, oxobenzthiazolinyl, benzoisothiazolyl, oxobenzoisothiazolinyl, benzimidazolyl, oxobenzimidazolinyl, indazolyl, oxoindazolinyl, benzofurazanyl, benzothiadiazolyl, benzotriazolyl, oxazolopyridyl, oxodihydrooxazolopyridyl, thiazolopyridyl, oxodihydrothiazolopyridyl, isothiazolopyridyl, imidazopyridyl, oxodihydroimidazopyridyl, pyrazolopyridyl, oxodihydropyrazolopyridyl, thienopyrimidinyl, chromanyl, chromanonyl, benzopyranyl, chromonyl, quinolyl, isoquinolyl, dihydroquinolyl, oxodihydroquinolinyl, tetrahydroquinolyl, oxotetrahydroquinolinyl, benzodioxanyl, quinoxalinyl, quinazolinyl, naphthyridinyl, carbazolyl, tetrahydrocarbazolyl, oxotetrahydrocarbazolyl, pyridoindolyl, acridinyl, oxodihydroacridinyl, phenothiazinyl, dihydrodibenzoxepinyl, oxodihydrodibenzoxepinyl, benzocycloheptathienyl, oxobenzocycloheptathienyl, dihydrothienobenzothiepinyl, oxodihydrothienobenzothiepinyl, dihydrodibenzothiepinyl, oxodihydrodibenzotbiepinyl, octahydrodibenzothiepinyl, dihydrodibenzazepinyl, oxodihydrodibenzazepinyl, octahydrodibenzazepinyl, benzocycloheptapyridyl, oxobenzocycloheptapyridyl, dihydropyridobenzodiazepinyl, dihydrodibenzoxazepinyl, dihydropyridobenzoxepinyl, dihydropyridobenzoxazepinyl, oxodihydropyridobenzoxazepinyl, dihydrodibenzothiazepinyl, oxodihydrodibenzothiazepinyl, dihydropyridobenzothiazepinyl, and oxodihydropyridobenzothiazepinyl, bound directly or over a methylene group,
R¹³ has the same meaning as R¹², but is selected independently therefrom,
R¹⁴ is selected from hydrogen, hydroxy, methyl, benzyl, phenyl, and,
   the group consisting of indanyl, indenyl, naphthyl, dihydronaphthyl, tetrahydronaphthyl, furyl, thienyl, pyrrolyl, oxazolyl, isoxazolyl, thiazolyl, isothiazolyl, pyrazolyl, imidazolyl, oxadiazolyl, thiadiazolyl, triazolyl, pyridyl, pyrazinyl, pyridazinyl, pyrimidinyl, triazinyl, benzofuryl, benzothienyl, indolyl, indolinyl, benzoxazolyl, benzothiazolyl, benzimidazolyl, chromanyl, quinolyl, and tetrahydroquinolyl, bound directly or over a methylene group,
G2 is selected from the residue

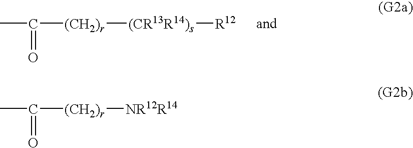

wherein the substituents R¹² and R¹⁴ have the above meanings, or the group

—NR¹²R¹⁴
is a nitrogen-containing heterocycle bound over the nitrogen atom, the nitrogen-containing heterocycle being selected from the group consisting of azetidine, pyrrolidine, piperidine, (1H)tetrahydropyridine, hexahydroazepine, (1H)tetrahydroazepine, octahydroazocine, pyrazolidine, piperazine, hexahydrodiazepine, morpholine, hexahydrooxazepine, thiomorpholine, thiomorpholine-1,1-dioxide, 5-aza-bicyclo[2.1.1]hexane, 2-aza-bicyclo[2.2.1]heptane, 7-aza-bicyclo[2.2.1]heptane, 2,5-diaza-bicyclo[2.2.1]-heptane, 2-aza-bicyclo[2.2.2]octane, 8-aza-bicyclo[3.2.1]octane, 2,5-diazabicyclo[2.2.2]octane, 9-azabicyclo[3.3.1]nonane, indoline, isoindoline, (1H)-dihydroquinoline, (1H)-tetrahydroquinoline, (2H)-tetrahydroisoquinoline, (1H)-tetrahydroquinoxaline, (4H)-dihydrobenzoxazine, (4H)-dihydrobenzothiazine, (1H)-tetrahydrobenzo[b]azepine, (1H)-tetrahydrobenzo[c]azepine, (1H)-tetrahydrobenzo[d]azepine, (5H)-tetrahydrobenzo[b]oxazepine, (5H)-tet-rahydrobenzo[b]thiazepine, 1,2,3,4-tetrahydro-9H-pyrido[3,4-b]indole, (10H)-dihydroacridine, 1,2,3,4-tetrahydroacridanone, (10H)-phenoxazine, (10H)-phenothiazine, (5H)-dibenzazepine, (5H)-dihydrodibenzazepine, (5H)-octahydrodibenzazepine, (5H)-dihydrodibenzodiazepine, (11H)-dihydrodibenzo[b,e]oxazepine, (11H)-dihydrodibenzo[b,e]thiazepine, (10H)-dihydrodibenzo[b,f]oxazepine, (10H)-dihydrodibenzo[b,f]thiazepine, and (5H)-tetrahydrodibenzazocine,
G3 is the residue

—SO₂—(CH₂)ᵣR¹² (G3),
G4 is the residue

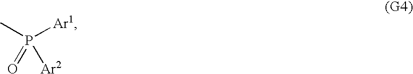

wherein
Ar¹ and Ar² are selected independently of each other from phenyl, pyridyl, and naphthyl,
G5 is the residue

—COR¹⁵ (G5)

wherein
R¹⁵ is selected from trifluoromethyl, C₁-C₆-alkoxy, C₃-C₆-alkenyloxy, and benzyloxy,
   wherein aromatic ring systems optionally are substituted independently of each other by one to three substituents independently selected from the group consisting of halogen, cyano, C₁-C₆-alkyl, trifluoromethyl, C₃-C₈-cycloalkyl, phenyl, benzyl, hydroxy, C₁-C₆-alkoxy, C₁-C₆-alkoxy entirely or partially substituted by fluorine; benzyloxy,
   phenoxy, mercapto, C₁-C₆-alkylthio, carboxy, C₁-C₆-alkoxycarbonyl, benzyloxycarbonyl, nitro, amino, mono-C₁-C₆-alkylamino, and di-(C₁-C₆-alkyl)-amino,

wherein two adjacent groups in the ring or ring system optionally form an additional ring over a methylenedioxy bridge.

In a further preferred embodiment, the inhibitor is a compound of formula (I), wherein:
R¹ is selected from hydrogen, halogen, cyano, methyl, trifluoromethyl, hydroxy, methoxy and methoxycarbonyl,
R² is hydrogen or halogen,
R³ is hydrogen,
R⁴ is selected from hydrogen, C₁-C₃-alkyl and hydroxy,
k is 0 or 1,
A is selected from C₂-C₆-alkenylene, optionally substituted once or twice by hydroxy or fluorine, or C₄-C₆-alkadienylene, optionally substituted by one or two fluorine atoms, and 1,3,5-hexatrienylene
D is selected from C₂-C₈-alkylene, optionally substituted by methyl or hydroxy
   C₂-C₈-alkenylene, optionally substituted by methyl or hydroxy, wherein the double bond optionally is to ring E, and
   the group consisting of C₂-C₈-alkylene and C₂-C₈-alkenylene, wherein one to three methylene units are isosterically replaced by O, NH, N(CH₃), N(COCH₃), N(SO₂CH₃) or CO,
E is selected from the residues

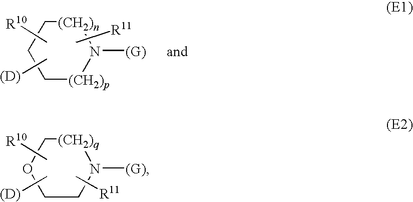

wherein the heterocyclic ring optionally has a double bond and
n and p are, independent of each other, 0, 1, 2 or 3, with the proviso that n+p≦3
q is 1 or 2,
R¹⁰ is selected from hydrogen, methyl and hydroxyl,
R¹¹ is hydrogen or an oxo group adjacent to the nitrogen atom,
G is selected from hydrogen, C₃-C₈-cycloalkyl, methoxycarbonyl, tert-butoxycarbonyl, benzyloxycarbonyl, trifluoroacetyl, diphenylphosphinoyl and the residues

—(CH₂)ᵣ—(CR¹³R¹⁴)ₛ—R¹² (G1)

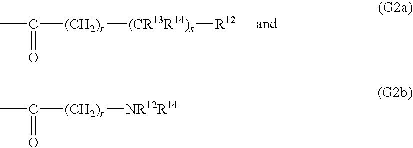

and

—SO₂—(CH₂)ᵣR¹² (G3)

wherein
r is 0, 1 or 2,
s is 0 or 1,
R¹² is selected from hydrogen, methyl, benzyl, phenyl.
   the group consisting of indanyl, indenyl, oxoindanyl, naphthyl, dihydronaphthyl, tetrahydronaphthyl, oxotetrahydronaphthyl, flourenyl, oxofluorenyl, anthryl, dihydroanthryl, oxodihydroanthryl, dioxodihydroanthryl, dibenzocycloheptenyl, and oxodiberizocycloheptenyl, dihydrodibenzocycloheptenyl, oxodihydrodibenzocycloheptenyl bound directly or over a methylene group, and
   the group consisting of furyl, thienyl, pyrrolyl, oxazolyl, isoxazolyl, thiazolyl, isothiazolyl, pyrazolyl, imidazolyl, oxadiazolyl, thiadiazolyl, triazolyl, pyridyl, pyrazinyl, pyridazinyl, pyrimidinyl, imidazothiazolyl, benzofuryl, dihydrobenzofuryl, benzothienyl, dihydrobenzothienyl, indolyl, indolinyl, oxoindolinyl, dioxoindolinyl, benzoxazolyl, oxobenzoxazolinyl, benzisoxazolyl, oxobenzisoxazolinyl, benzothiazolyl, oxobenzthiazolinyl, benzoisothiazolyl, oxobenzoisothiazolinyl, benzimidazolyl, oxobenzimidazolinyl, benzofurazanyl, benzothiadiazolyl, benzotriazolyl, oxazolopyridyl, oxodihydrooxazolopyridyl, thiazolopyridyl, oxodihydrothiazolopyridyl, isothiazolopyridyl, imidazopyridyl, oxodihydroimidazopyridyl, pyrazolopyridyl, thienopyrimidinyl, chromanyl, chromanonyl, benzopyranyl, chromonyl, quinolyl, isoquinolyl, dihydroquinolyl, oxodihydroquinolinyl, tetrahydroquinolyl, oxotetrahydroquinolinyl, benzodioxanyl, quinoxalinyl, quinazolinyl, naphthyridinyl, carbazolyl, tetrahydrocarbazolyl, oxotetrahydrocarbazolyl, pyridoindolyl, acridinyl, oxodihydroacridinyl, phenothiazinyl, dihydrodibenzoxepinyl, benzocycloheptathienyl, oxobenzocycloheptathienyl, dihydrothienobenzothiepinyl, oxodihydrothienobenzothiepinyl, dihydrodibenzothiepinyl, oxodihydrodibenzothiepinyl, dihydrodibenzazepinyl, oxodihydrodibenzazepinyl, octahydrodibenzazepinyl, benzocycloheptapyridyl, oxobenzocycloheptapyridyl, dihydropyridobenzoxepinyl, dihydrodibenzothiazepinyl, and oxodihydrodibenzothiazepinyl, bound directly or over a methylene group,
R¹³ is selected from hydrogen, methyl, benzyl and phenyl,
R¹⁴ is selected from hydrogen, hydroxy, methyl, benzyl, phenyl, and
   the group consisting of naphthyl, furyl, thienyl, oxazolyl, thiazolyl, pyrazolyl, imidazolyl, oxadiazolyl, thiadiazolyl, pyridyl, benzofuryl, benzothienyl, indolyl, indolinyl, benzoxazolyl, benzothiazolyl, benzimidazolyl, chromanyl, quinolyl and tetrahydroquinolyl, bound directly or over a methylene group, wherein in formula

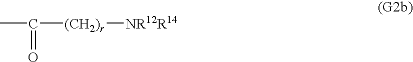

—NR¹²R¹⁴ optionally is selected from pyrrolidine, piperidine, (1H)-tetrahydropyridine, hexahydroazepine, octahydroazocine, piperazine, hexahydrodiazepine, morpholine, hexahydrooxazepine, 2-azabicyclo[2.2.1]heptane, 7-azabicyclo[2.2.1]heptane, 2,5-diazabicyclo[2.2.1]heptane, 8-azabicyclo[3.2.1]octane, 2,5-diazabicyclo[2.2.2]octane, indoline, isoindoline, (1H)-dihydroquinoline, (1H)-tetrahydroquinoline, (2H)-tetrahydroisoquinoline, (1H)-tetrahydroquinoxaline, (4H)-dihydrobenzoxazine, (4H)-dihydrobenzothiazine, (1H)-tetrahydrobenzo[b]azepine, (1H)-tetrahydrobenzo[d]azepine, (5H)-tetrahydrobenzo[b]oxazepine, (5H)-tetrahydrobenzo[b]thiazepine, 1,2,3,4-tetrahydro-9H-pyrido[3,4-b]indol, (10H)-dihydroacridine, 1,2,3,4-tetrahydroacridanone, (5H)-dihydrodibenzazepine, (5H)-dihydrodibenzodiazepine, (11H)-dihydrodibenzo[b,e]oxazepine, (11H)-dihydrodibenzo[b,e]thiazepine, (10H)-dihydrodibenzo[b,f]oxazepine and (5H)-tetrahydrodibenzazocine
   In an even further preferred embodiment according to the invention, the inhibitor is a compound of formula (I), wherein:
R¹ is selected from hydrogen, fluorine, chlorine, bromine, methyl, trifluoromethyl and hydroxy,
R² and
R³ are hydrogen,
R⁴ is hydrogen or hydroxy,
k is 0 or 1,
A is C₂-C₄-alkenylene, which is optionally substituted by fluorine,
D is selected from C₂-C₆-alkylene, C₂-C₆-alkenylene, wherein the double bond optionally is to ring E, and the group consisting of C₂-C₆-alkylene and C₂-C₆-alkenylene, wherein a methylene unit is isosterically replaced by O, NH, N(CH₃) or CO, or an ethylene group is isosterically replaced by NH—CO or CO—NH, or a propylene group is isosterically replaced by NH—CO—O or O—CO—NH,
E is selected from pyrrolidine, piperidine, 1,2,5,6-tetrahydropyridine, hexahydroazepine, morpholine and hexahydro-1,4-oxazepine, wherein the heterocyclic ring optionally is substituted by an oxo group adjacent to the nitrogen atom,
G is selected from hydrogen, tert-butoxycarbonyl, diphenylphosphinoyl, and one of the residues

—(CH₂)ᵣ—(CR¹³R¹⁴)ₛ—R¹² (G1)

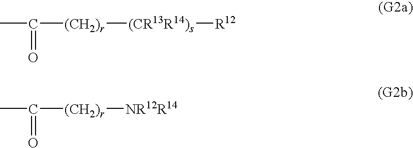

and

—SO₂—(CH₂)ᵣR¹² (G3)

wherein
r is 0 or 1,
s is 0 or 1,
R¹² is selected from hydrogen, methyl, benzyl, phenyl.
   the group consisting of indenyl, oxoindanyl, naphthyl, tetrahydronaphthyl, flourenyl, oxofluorenyl, anthryl, dihydroanthryl, oxodihydroanthryl, dioxodihydroanthryl and dibenzocycloheptenyl, dihydrodibenzocycloheptenyl, bound directly or over a methylene group, and
   the group consisting of furyl, thienyl, oxazolyl, thiazolyl, imidazolyl, oxadiazolyl, thiadiazolyl, pyridyl, pyrazinyl, pyrimidinyl, imidazothiazolyl, benzofuryl, benzothienyl, indolyl, oxoindolinyl, dioxoindolinyl, benzoxazolyl, oxobenzoxazolinyl, benzothiazolyl, oxobenzthiazolinyl, benzimidazolyl, oxobenzimidazolinyl, benzofurazanyl, benzotriazolyl, oxazolopyridyl, oxodihydrooxazolopyridyl, thiazolopyridyl, oxodihydrothiazolopyridyl, chromanyl, chromanonyl, benzopyranyl, chromonyl, quinolyl, isoquinolyl, oxodihydroquinolinyl, tetrahydroquinolyl, oxotetrahydroquinolinyl, benzodioxanyl, quinazolinyl, acridinyl, oxodihydroacridinyl, phenothiazinyl, dihydrodibenzoxepinyl, benzocycloheptathienyl, dihydrothienobenzothiepinyl, dihydrodibenzothiepinyl, oxodihydrodibenzothiepinyl, dihydrodibenzazepinyl, oxodihydrodibenzazepinyl, octahydrodibenzazepinyl, benzocycloheptapyridyl, oxobenzocycloheptapyridyl, and dihydrodibenzothiazepinyl, bound directly or over a methylene group,
R¹³ is selected from hydrogen, methyl, benzyl and phenyl,
R¹⁴ is selected from hydrogen, hydroxy, methyl, benzyl, phenyl, and
   the group consisting of naphthyl, furyl, thienyl, pyridyl, benzofuryl, benzothienyl, indolyl, benzoxazolyl, benzothiazolyl, benzimidazolyl, chromanyl, quinolyl and tetrahydroquinolyl, bound directly or over a methylene group,
   wherein in formula

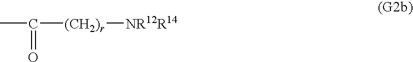

—NR¹²R¹⁴ optionally is selected from pyrrolidine, piperidine, hexahydroazepine, morpholine, 2,5-diazabicyclo[2.2.1]heptane, indoline, isoindoline, (1H)-dihydroquinoline, (1H)-tetrahydroquinoline, (2H)-tetrahydroisoquinoline, (1H)-tetrahydrobenzo[b]azepine, (1H)-tetrahydrobenzo[d]azepine, (5H)-tetrahydrobenzo[b]oxazepine, (5H)-tetrahydrobenzo[b]thiazepine, 1,2,3,4-tetrahydroacridanone, (5H)-dihydrodibenzazepine, (11H)-dihydrodibenzo[b,e]oxazepine, and (11H)-dihydrodibenzo[b,e]thiazepine,
   wherein aromatic ring systems optionally are substituted, independently of each other, by one to three substituents which are independently selected from the group consisting of halogen, cyano, C₁-C₆-alkyl, trifluoromethyl, C₃-C₈-cycloalkyl, phenyl, benzyl, hydroxy, C₁-C₆-alkoxy, C₁-C₆-alkoxy which is entirely or partially substituted by fluorine; benzyloxy, phenoxy, mercapto, C₁-C₆-alkylthio, carboxy, C₁-C₆-alkoxycarbonyl, benzyloxycarbonyl, nitro, amino, mono-C₁-C₆-alkylamino and di-(C₁-C₆-alkyl)-amino, wherein two adjacent groups on the aromatic ring or ring system optionally form an additional ring over a methylenedioxy bridge.

An especially preferred embodiment according to the invention relates to compounds of formula (I), wherein:
R¹ is selected from hydrogen, fluorine, methyl, trifluoromethyl and hydroxy,
R² and
R³ are hydrogen,
R⁴ is hydrogen or hydroxy,
k is 0,
A is ethenylene or 1,3-butadienylene
D is C₂-C₆-alkylene or C₂-C₆-alkenylene, wherein the double bond optionally is to ring E,
E is selected from pyrrolidine, piperidine, hexahydroazepine and morpholine,
G is selected from benzyl, phenethyl, fluorenylmethyl, anthrylmethyl, diphenylmethyl, fluorenyl, dihydrodibenzocycloheptenyl, furylmethyl, thienylmethyl, thiazolylmethyl, pyridylmethyl, benzothienylmethyl, quinolylmethyl, phenyl-thienylmethyl phenyl-pyridylmethyl, dihydrodibenzoxepinyl, dihydrodibenzothiepinyl,
   acetyl, pivaloyl, phenylacetyl, diphenylacetyl, diphenylpropionyl, naphthylacetyl, benzoyl, naphthoyl, anthrylcarbonyl, oxofluorenylcarbonyl, oxodihydroanthrylcarbonyl, dioxodihydroanthrylcarbonyl, furoyl, pyridylcarbonyl, chromonylcarbonyl, quinolylcarbonyl, naphthylaminocarbonyl, dibenzylaminocarbonyl, benzylphenylaminocarbonyl, diphenylaminocarbonyl, indolinyl-1-carbonyl, dihydrodibenzazepin-N-carbonyl, tetrahydroquinolinyl-N-carbonyl, tetrahy-drobenzo[b]azepinyl-N-carbonyl,
   methanesulfonyl, phenylsulfonyl, p-toluenesulfonyl, naphthylsulfonyl, quinolinsulfonyl, and diphenylphosphinoyl,
      wherein aromatic ring systems optionally are substituted independently of each other by one to three substituents which are independently selected from the group consisting of halogen, cyano, C₁-C₆-alkyl, trifluoromethyl, C₃-C₈-cycloalkyl, phenyl, benzyl, hydroxy, C₁-C₆-alkoxy, C₁-C₆-alkoxy, entirely or partially substituted by fluorine; benzyloxy, phenoxy, mercapto, C₁-C₆-alkylthio, carboxy, C₁-C₆-alkoxycarbonyl, benzyloxycarbonyl, nitro, amino, mono-C₁-C₆-alkylamino and di-(C₁-C₆-alky)-amino, wherein two adjacent groups in the ring or ring system optionally form an additional ring over a methylenedioxy bridge.

A series of exemplary compounds with the respective substituent definitions are listed in the following Table for illustration of the invention.

TABLE 1
Exemplifying compounds of
formula (I) according to the
invention

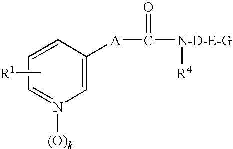

| Nr | R¹ | k | A | R⁴ | D-E-G |
|---|---|---|---|---|---|
| 1 | H | 0 | CH═CH | H | 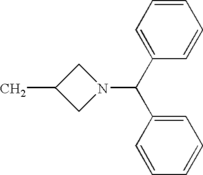 |
| 2 | H | 0 | CH═CH—CH═CH | H | 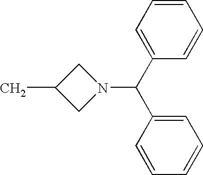 |
| 3 | H | 0 | CH═CH | H | 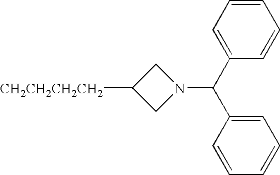 |
| 4 | H | 0 | CH═CH | H | 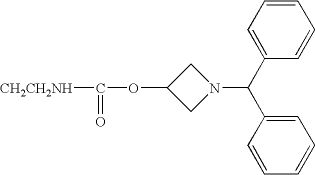 |
| 5 | H | 0 | CH═CH | H | 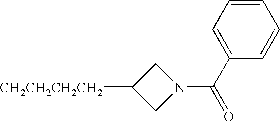 |
| 6 | H | 0 | CH═CH | H | 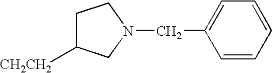 |
| 7 | H | 0 | CH═CH—CH═CH | H | 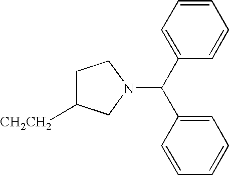 |
| 8 | H | 0 | CH═CH(CH₂)₂ | H | 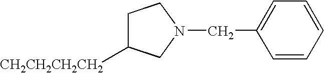 |
| 9 | H | 0 | CH═CH | H | 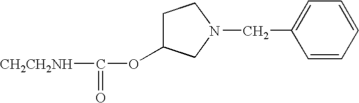 |
| 10 | H | 0 | CH═CH | H | 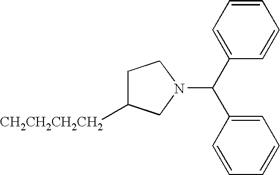 |
| 11 | H | 0 | CH═CH | H | 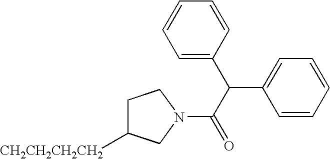 |
| 12 | H | 0 | CH═CH | H | 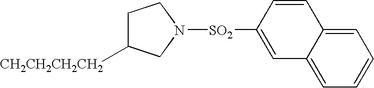 |
| 13 | H | 0 | CH═CH | H | 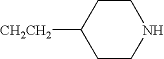 |
| 14 | H | 0 | CH═CH | H | 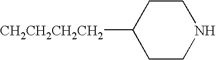 |
| 15 | H | 0 | CH═CH—CH═CH | H | 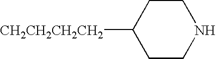 |
| 16 | H | 0 | CH═CH | H | 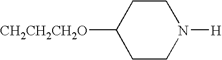 |
| 17 | H | 0 | CH═CH | H | 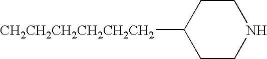 |
| 18 | H | 0 | CH═CH—CH═CH | H | 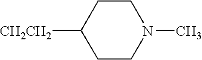 |
| 19 | H | 0 | CH═CH—CH═CH | H | 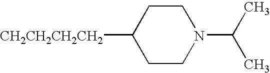 |
| 20 | H | 0 | CH═CH(CH₂)₂ | H | 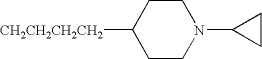 |
| 21 | H | 0 | CH═CH | H | 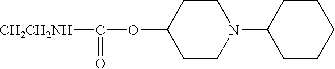 |
| 22 | H | 0 | 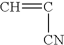 | H | 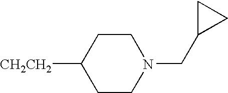 |
| 23 | H | 0 | CH═CH | H | 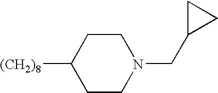 |
| 24 | H | 0 | CH═CH | H | 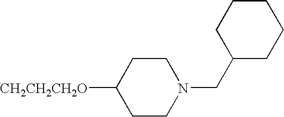 |
| 25 | H | 0 | CH═CH | H | 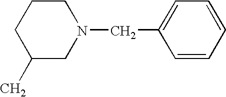 |
| 26 | H | 0 | CH═CH | H | 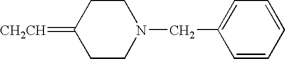 |
| 27 | H | 0 | CH═CH—CH═CH | H | 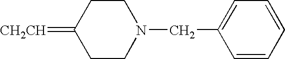 |
| 28 | H | 1 | CH═CH | H | 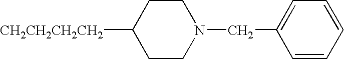 |
| 29 | H | 0 | CH═CH | OH | 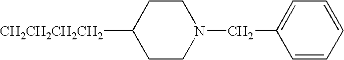 |
| 30 | H | 0 | 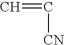 | H | 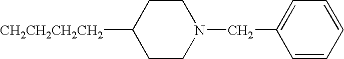 |
| 31 | H | 0 | C≡C | H | 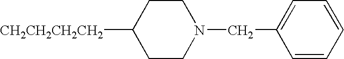 |
| 32 | H | 0 | CH═CH(CH₂)₂ | H | 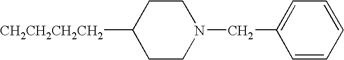 |
| 33 | H | 0 | CH═CH—CH═CH | H | 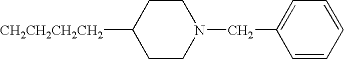 |
| 34 | 2-F | 0 | CH═CH—CH═CH | H | 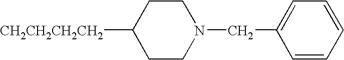 |
| 35 | H | 0 | (CH═CH)₃ | H | 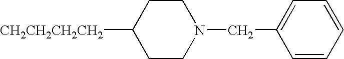 |
| 36 | H | 0 | CH═CH | H | 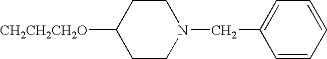 |
| 37 | H | 0 | CH═CH | H | 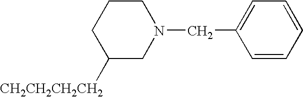 |
| 38 | H | 0 | CH═CH | H | 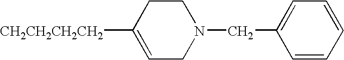 |
| 39 | H | 0 | CH═CH | H | 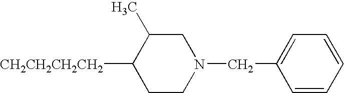 |
| 40 | H | 0 | CH═CH | H | 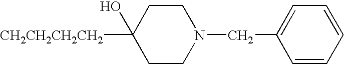 |
| 41 | H | 0 | CH═CH | H | 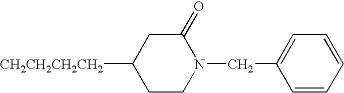 |
| 42 | H | 0 | CH═CH—CH═CH | H | 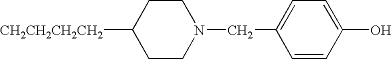 |
| 43 | H | 0 | CH═CH—CH═CH | H | 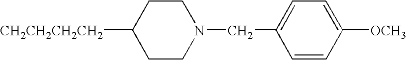 |
| 44 | H | 0 | CH═CH—CH═CH | H | 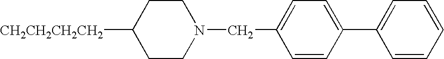 |
| 45 | H | 0 | CH═CH—CH═CH | H | 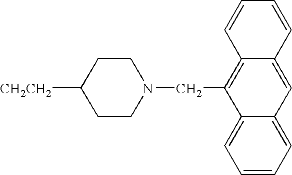 |
| 46 | H | 0 | C≡C | H | 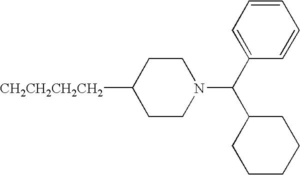 |
| 47 | H | 0 | CH═CH | H | 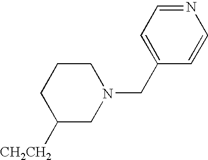 |
| 48 | H | 0 | CH═CH—CH═CH | H | 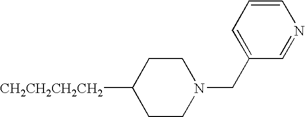 |
| 49 | H | 0 | CH═CH | H | 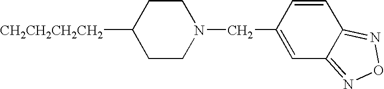 |
| 50 | H | 0 | CH═CH | H | 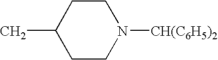 |
| 51 | H | 0 | CH═CH—CH═CH | H | 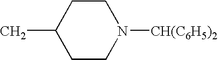 |
| 52 | H | 1 | CH═CH | H | 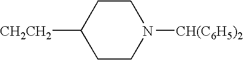 |
| 53 | H | 0 | CH═CH(CH₂)₂ | H | 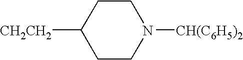 |
| 54 | H | 0 | CH═CHCH₂CHF | H | 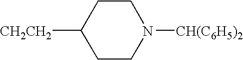 |
| 55 | H | 0 | CH═CH | H | 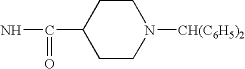 |
| 56 | H | 0 | CH═CH | H | 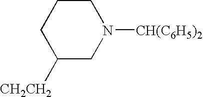 |
| 57 | H | 0 | CH═CH—CH═CH | H | 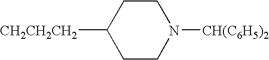 |
| 58 | H | 0 | CH═CH | H | 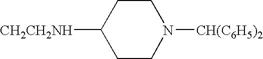 |
| 59 | H | 1 | CH═CH | H | 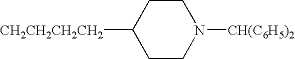 |
| 60 | H | 0 | CH═CH | OH | 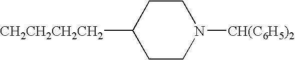 |
| 61 | H | 0 | 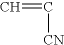 | H | 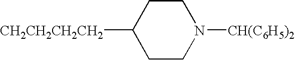 |
| 62 | H | 0 | C≡C | H | 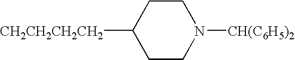 |
| 63 | H | 0 | CH═CH(CH₂)₂ | H | 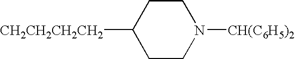 |
| 64 | H | 0 | 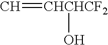 | H | 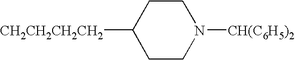 |
| 65 | H | 0 | (CH₂)₂CH═CH | H | 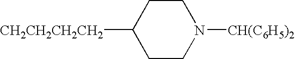 |
| 66 | H | 0 | CH═CH—CH═CH | H | 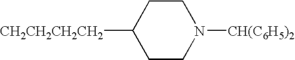 |
| 67 | H | 0 | CH═CH—CH═CH | CH₃ | 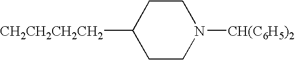 |
| 68 | 2-F | 0 | CH═CH—CH═CH | H | 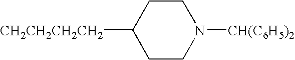 |
| 69 | 2-F | 0 | CH═CH—CH═CH | OH | 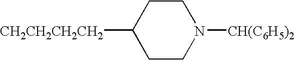 |
| 70 | 4-F | 0 | CH═CH—CH═CH | H | 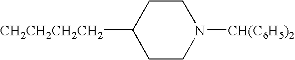 |
| 71 | 5-F | 0 | CH═CH—CH═CH | H | 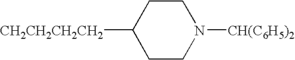 |
| 72 | 6-F | 0 | CH═CH—CH═CH | H | 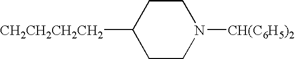 |
| 73 | 2-Cl | 0 | CH═CH—CH═CH | H | 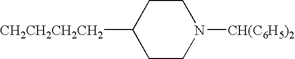 |
| 74 | 6-CH₃ | 0 | CH═CH—CH═CH | H | 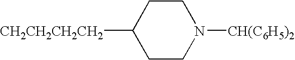 |
| 75 | 2-OH | 0 | CH═CH—CH═CH | H | 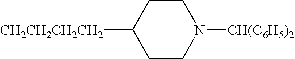 |
| 76 | H | 0 | (CH═CH)₃ | H | 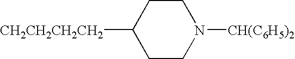 |
| 77 | H | 0 | CH═CH | H | 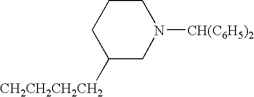 |
| 78 | 2-F | 0 | CH═CH | H | 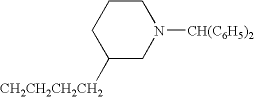 |
| 79 | 5-F | 0 | CH═CH | H | 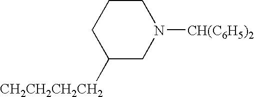 |
| 80 | 6-CH₃O | 0 | CH═CH | H | 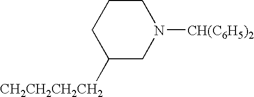 |
| 81 | H | 0 | CH═CH—CH═CH | H | 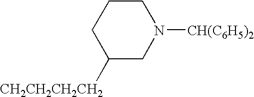 |
| 82 | H | 0 | CH═CH | H | 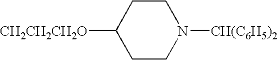 |
| 83 | H | 0 | CH═CH—CH═CH | H | 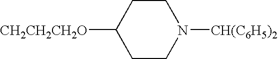 |
| 84 | H | 0 | CH═CH | H | 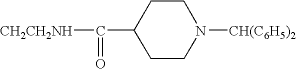 |
| 85 | H | 0 | CH═CH | H | 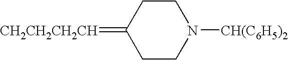 |
| 86 | H | 0 | CH═CH | H | 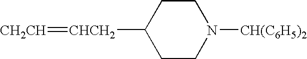 |
| 87 | H | 0 | CH═CH | H | 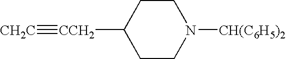 |
| 88 | H | 0 | CH═CH | H | 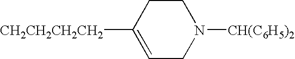 |
| 89 | H | 0 | CH═CH—CH═CH | H | 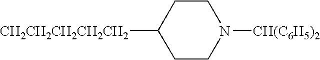 |
| 90 | H | 0 | CH═CH | H | 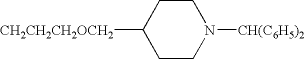 |
| 91 | H | 0 | CH═CH—CH═CH | H | 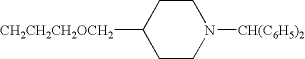 |
| 92 | H | 0 | CH═CH | H | 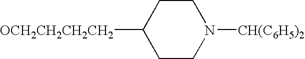 |
| 93 | H | 0 | CH═CH | H | 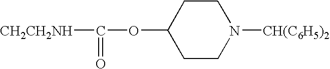 |
| 94 | H | 0 | CH═CH—CH═CH | H | 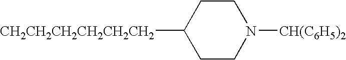 |
| 95 | H 96 | 0 | CH═CH | H | 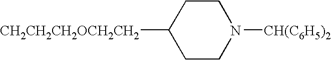 |
| 96 | H | 0 | CH═CH | H | 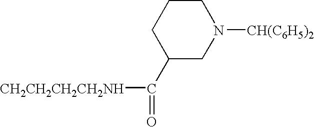 |
| 97 | H | 0 | CH═CH | H | 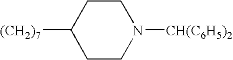 |
| 98 | H | 0 | CH═CH | H | 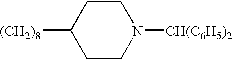 |
| 99 | H | 0 | CH═CH | H | 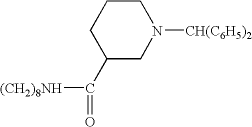 |
| 100 | H | 0 | CH═CH | H | 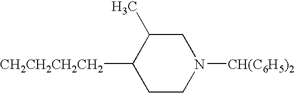 |
| 101 | H | 0 | CH═CH | H | 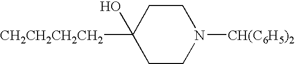 |
| 102 | H | 0 | CH═CH | H | 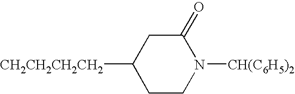 |
| 103 | H | 0 | CH═CH—CH═CH | H | 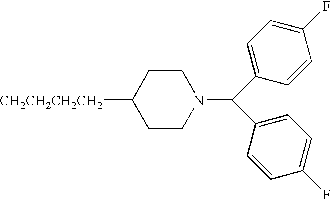 |
| 104 | H | 0 | C≡C | H | 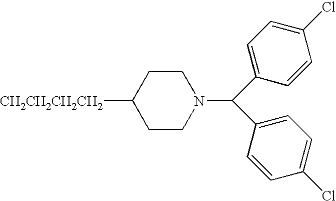 |
| 105 | H | 0 | CH═CH—CH═CH | H | 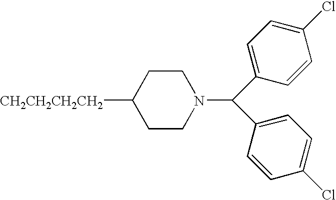 |
| 106 | H | 0 | C≡C | H | 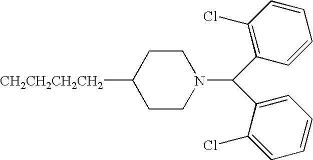 |
| 107 | H | 0 | (CH₂)₂CH ═CH | H | 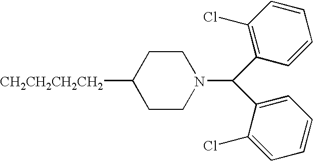 |
| 108 | H | 0 | CH═CH—CH═CH | H | 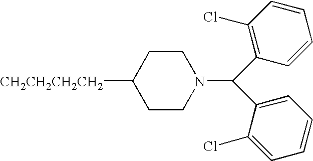 |
| 109 | H | 0 | CH═CH—CH═CH | H | 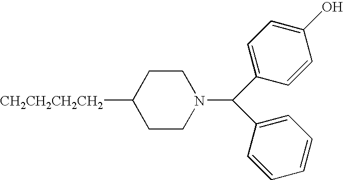 |
| 110 | H | 0 | CH═CH—CH═CH | H | 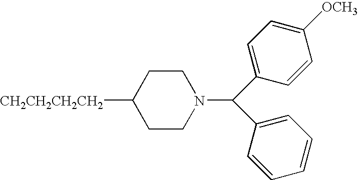 |
| 111 | H | 0 | CH═CH—CH═CH | H | 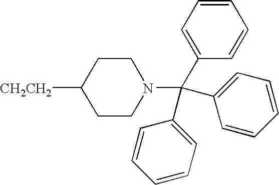 |
| 112 | H | 0 | CH═CH—CH═CH | H | 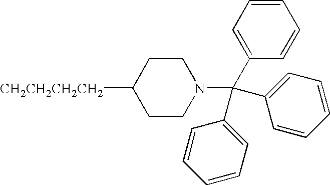 |
| 113 | H | 0 | CH═CH | H | 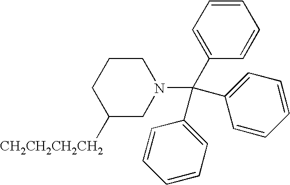 |
| 114 | H | 0 | CH═CH—CH═CH | H | 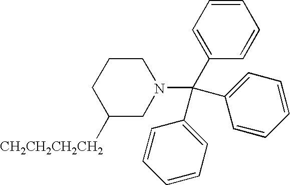 |
| 115 | H | 0 | CH═CH | H | 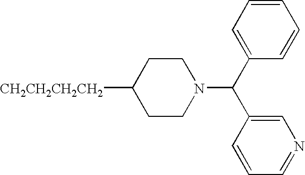 |
| 116 | H | 0 | CH═CH—CH═CH | H | 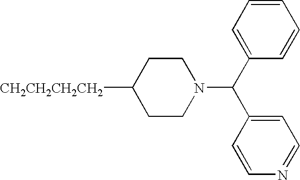 |
| 117 | H | 0 | CH═CH | H | 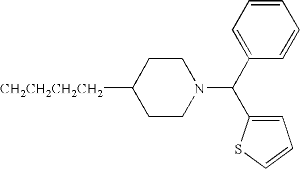 |
| 118 | H | 0 | CH═CH—CH═CH | H | 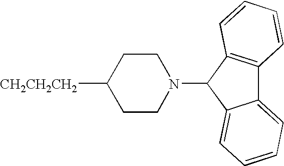 |
| 119 | H | 0 | 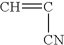 | H | 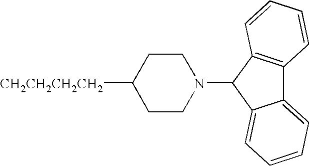 |
| 120 | H | 0 | CH═CH—CH═CH | H | 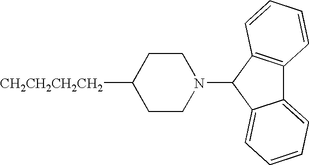 |
| 121 | H | 0 | CH═CHCH₂CHF | H | 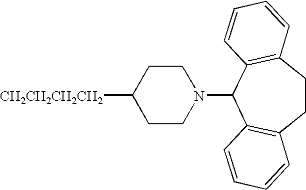 |
| 122 | H | 0 | CH═CH—CH═CH | H | 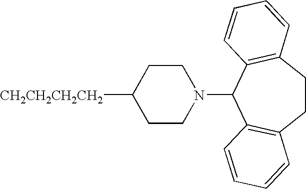 |
| 123 | H | 0 | C≡C | H | 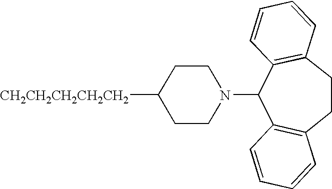 |
| 124 | H | 0 | CH═CH | H | 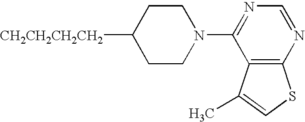 |
| 125 | H | 0 | CH═CH—CH═CH | H | 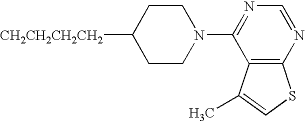 |
| 126 | H | 0 | CH═CH | H | 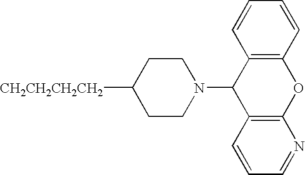 |
| 127 | H | 0 | CH═CH | H | 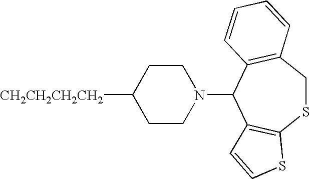 |
| 128 | H | 0 | CH═CH | H | 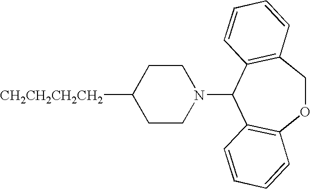 |
| 129 | H | 0 | CH═CH—CH═CH | H | 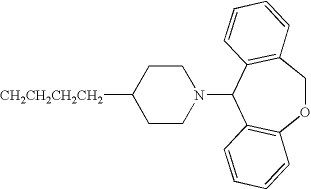 |
| 130 | H | 0 | CH═CH | H | 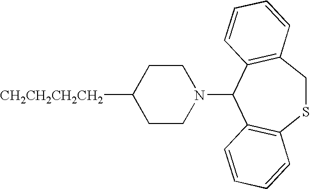 |
| 131 | H | 0 | CH═CH—CH═CH | H | 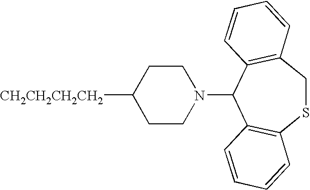 |
| 132 | H | 0 | CH═CH | H | 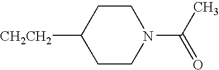 |
| 133 | H | 0 | CH═CH—CH═CH | H | 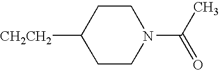 |
| 134 | H | 0 | CH═CH | H | 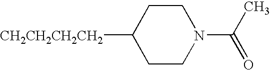 |
| 135 | H | 0 | CH═CH—CH═CH | H | 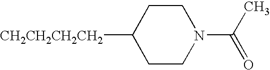 |
| 136 | H | 0 | CH═CH | H | 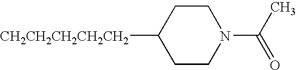 |
| 137 | H | 0 | CH═CH | H | 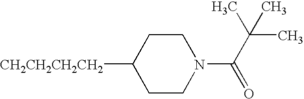 |
| 138 | H | 0 | CH═CH | H | 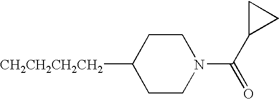 |
| 139 | H | 0 | CH═CH—CH═CH | H | 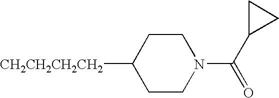 |
| 140 | H | 0 | CH═CH | H | 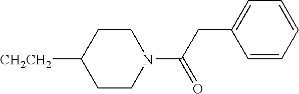 |
| 141 | H | 0 | CH═CH | H | 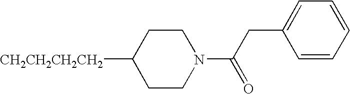 |
| 142 | H | 0 | CH═CH—CH═CH | H | 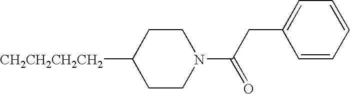 |
| 143 | H | 0 | CH═CH(CH₂)₂ | H | 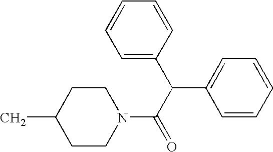 |
| 144 | H | 0 | CH═CH | H | 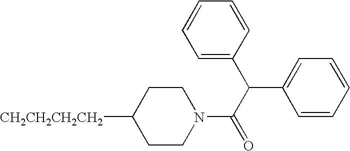 |
| 145 | H | 0 | CH═CH—CH═CH | H | 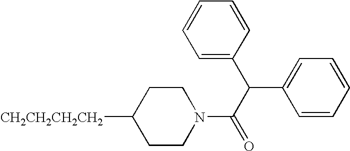 |
| 146 | H | 0 | CH═CH | H | 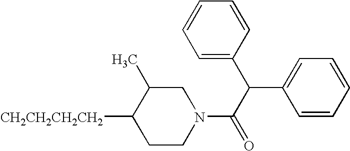 |
| 147 | H | 0 | CH═CH | H | 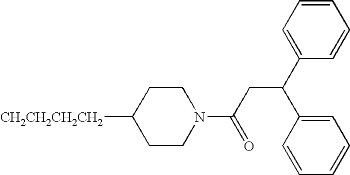 |
| 148 | H | 0 | CH═CH—CH═CH | H | 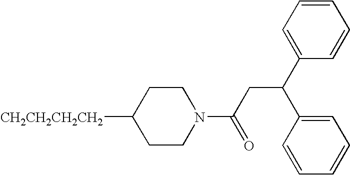 |
| 149 | H | 0 | CH═CH | H | 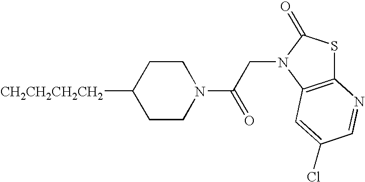 |
| 150 | H | 0 | CH═CH—CH═CH | H | 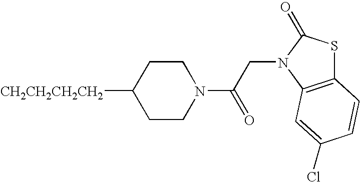 |
| 151 | H | 0 | CH═CH | H | 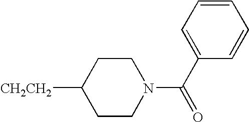 |
| 152 | H | 0 | CH═CH | H | 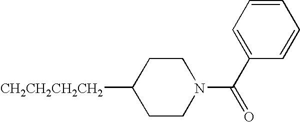 |
| 153 | H | 0 | 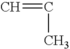 | H | 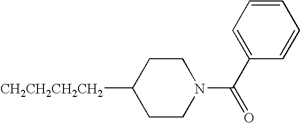 |
| 154 | H | 0 | 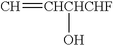 | H | 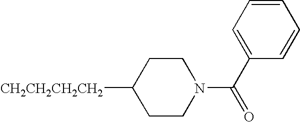 |
| 155 | H | 0 | CH═CH—CH═CH | H | 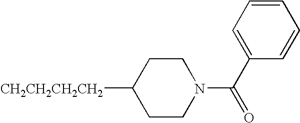 |
| 156 | H | 0 | CH═CH | H | 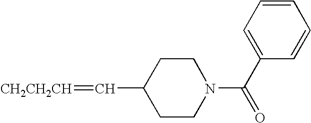 |
| 157 | H | 0 | CH═CH | CH₃ | 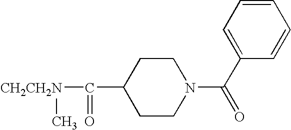 |
| 158 | H | 0 | CH═CH | H | 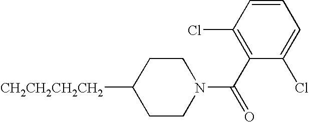 |
| 159 | H | 0 | CH═CH | H | 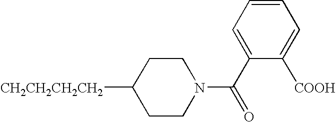 |
| 160 | H | 0 | CH═CH | H | 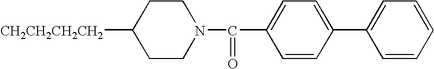 |
| 161 | H | 0 | CH═CH—CH═CH | H | 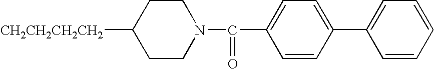 |
| 162 | H | 0 | CH═CH | H | 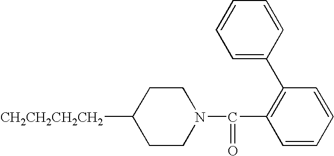 |
| 163 | H | 0 | (CH₂)₂CH═CH | H | 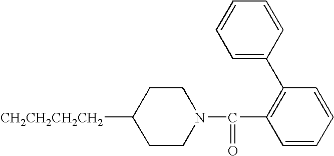 |
| 164 | H | 0 | CH═CH | H | 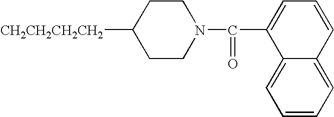 |
| 165 | 2-F | 0 | CH═CH | H | 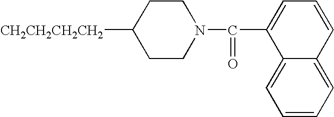 |
| 166 | H | 0 | CH═CH—CH═CH | H | 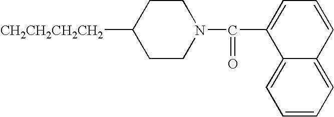 |
| 167 | H | 0 | CH═CH | H | 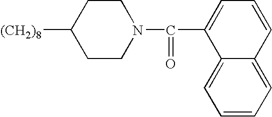 |
| 168 | H | 0 | CH═CH | H | 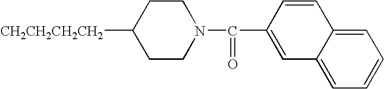 |
| 169 | H | 0 | CH═CH—CH═CH | H | 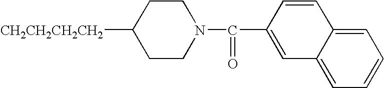 |
| 170 | H | 0 | CH═CH | H | 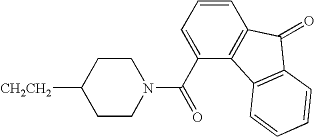 |
| 171 | H | 0 | CH═CH | H | 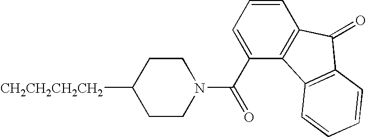 |
| 172 | H | 0 | CH═CH—CH═CH | H | 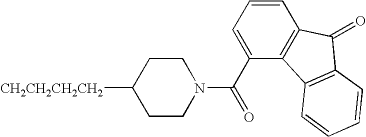 |
| 173 | H | 0 | CH═CH | H | 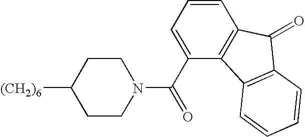 |
| 174 | H | 0 | CH═CH | H | 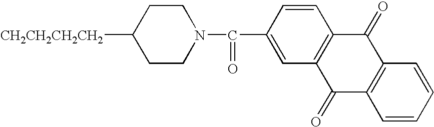 |
| 175 | 4-F | 0 | CH═CH | H | 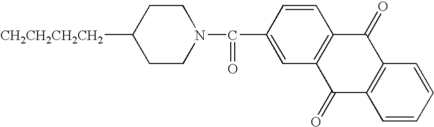 |
| 176 | H | 0 | CH═CH—CH═CH | H | 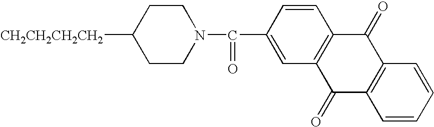 |
| 177 | H | 0 | CH═CH | H | 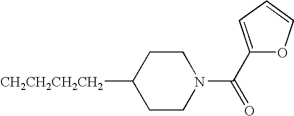 |
| 178 | H | 0 | CH═CH | H | 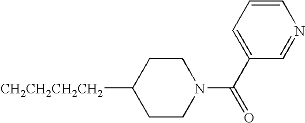 |
| 179 | H | 0 | (CH═CH)₃ | H | 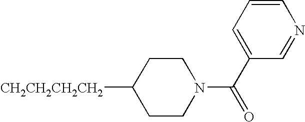 |
| 180 | H | 0 | CH═CH | H | 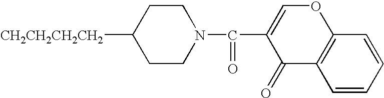 |
| 181 | H | 0 | CH═CH—CH═CH | H | 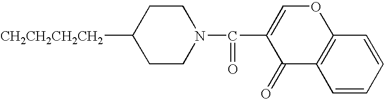 |
| 182 | H | 0 | CH═CH | H | 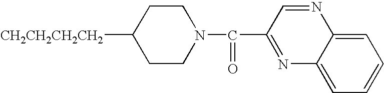 |
| 183 | H | 0 | C≡C | H | 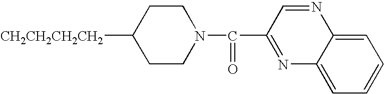 |
| 184 | H | 0 | CH═CH | H | 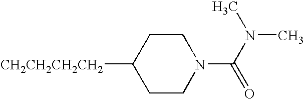 |
| 185 | H | 0 | CH═CH | H | 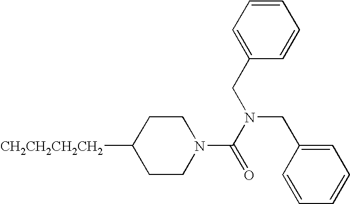 |
| 186 | H | 0 | CH═CH—CH═CH | H | 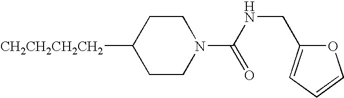 |
| 187 | H | 0 | CH═CH | H | 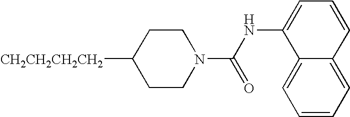 |
| 188 | 2-Cl | 0 | CH═CH | H | 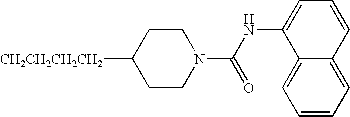 |
| 189 | H | 0 | CH═CH | H | 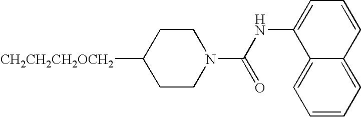 |
| 190 | H | 0 | CH═CH | H | 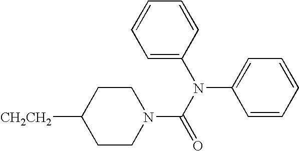 |
| 191 | H | 0 | CH═CH | H | 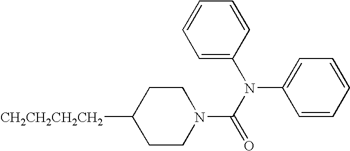 |
| 192 | H | 0 | CH═CH—CH═CH | H | 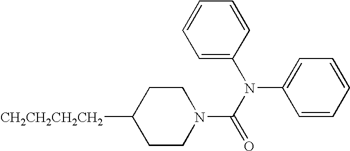 |
| 193 | H | 0 | CH═CH | H | 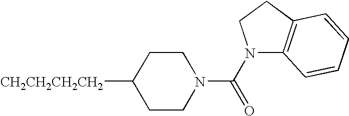 |
| 194 | H | 0 | CH═CH—CH═CH | H | 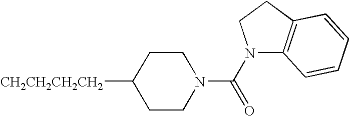 |
| 195 | H | 0 | CH═CH | H | 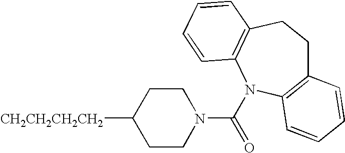 |
| 196 | H | 0 | CH═CH—CH═CH | H | 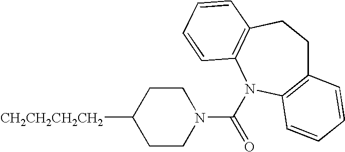 |
| 197 | H | 0 | CH═CH | H | 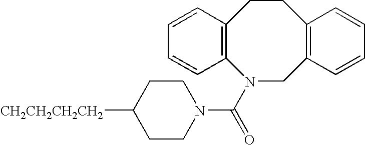 |
| 198 | H | 0 | CH═CH—CH═CH | H | 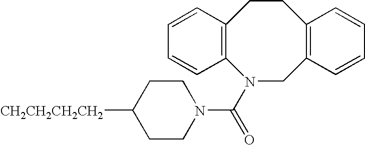 |
| 199 | H | 0 | CH═CH | H | 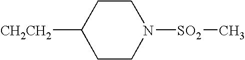 |
| 200 | H | 0 | CH═CH | H | 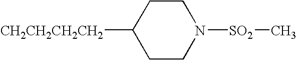 |
| 201 | H | 0 | CH═CH—CH═CH | H | 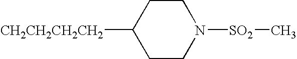 |
| 202 | H | 0 | CH═CH | H | 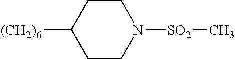 |
| 203 | H | 0 | CH═CH | H | 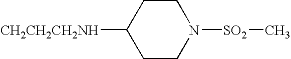 |
| 204 | H | 0 | CH═CH | H | 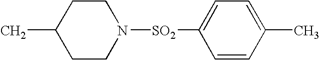 |
| 205 | H | 0 | CH═CH | H | 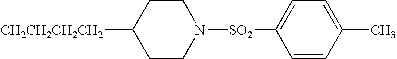 |
| 206 | H | 0 | CH═CH—CH═CH | H | 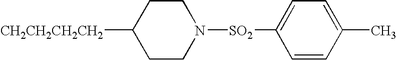 |
| 207 | H | 0 | CH═CH | H | 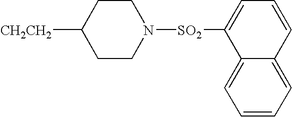 |
| 208 | H | 0 | CH═CH | H | 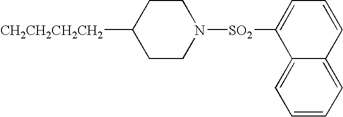 |
| 209 | H | 0 | CH═CH | H | 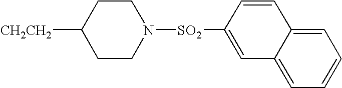 |
| 210 | H | 0 | CH═CH—CH═CH | H | 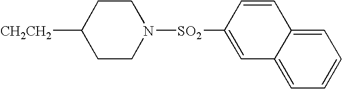 |
| 211 | H | 0 | CH═CH | H | 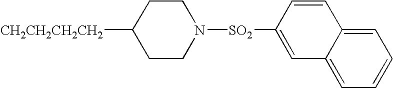 |
| 212 | H | 0 | C≡C | H | 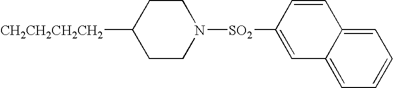 |
| 213 | H | 0 | (CH₂)₂CH═CH | H | 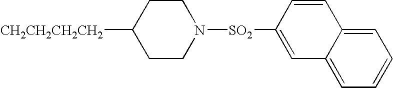 |
| 214 | H | 0 | CH═CH—CH═CH | H | 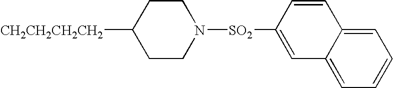 |
| 215 | H | 0 | CH═CH | H | 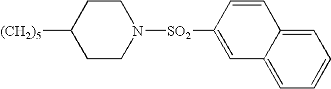 |
| 216 | H | 0 | CH═CH | H | 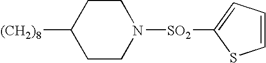 |
| 217 | H | 0 | CH═CH | H | 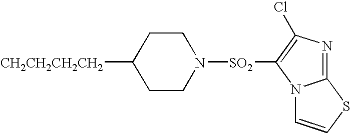 |
| 218 | H | 0 | CH═CH—CH═CH | H | 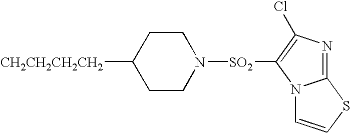 |
| 219 | H | 0 | CH═CH | H | 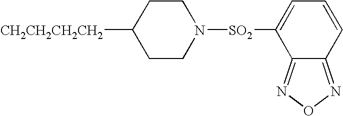 |
| 220 | H | 0 | CH═CH | H | 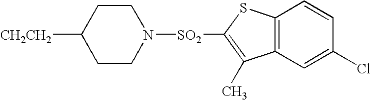 |
| 221 | H | 0 | CH═CH | H | 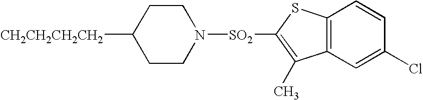 |
| 222 | H | 0 | CH═CH—CH═CH | H | 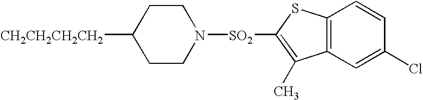 |
| 223 | H | 0 | CH═CH | H | 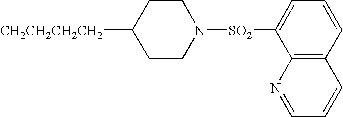 |
| 224 | H | 0 | CH═CH | H | 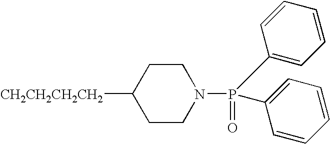 |
| 225 | H | 0 | CH═CH—CH═CH | H | 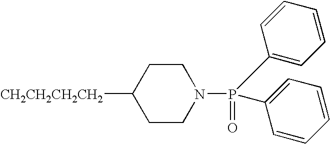 |
| 226 | H | 0 | CH═CH | H | 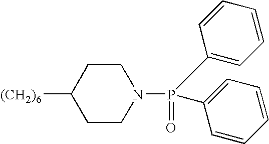 |
| 227 | H | 0 | CH═CH | H | 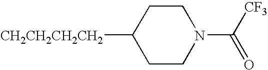 |
| 228 | H | 0 | CH═CH—CH═CH | H | 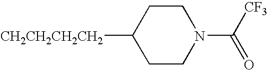 |
| 229 | H | 0 | CH═CH | H | 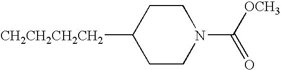 |
| 230 | H | 0 | CH═CH—CH═CH | H | 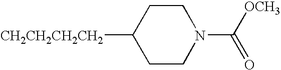 |
| 231 | H | 0 | CH═CH | H | 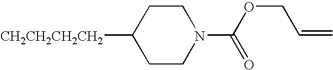 |
| 232 | H | 0 | CH═CH | H | 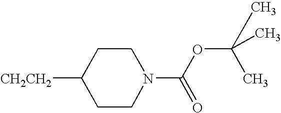 |
| 233 | H | 0 | CH═CH—CH═CH | H | 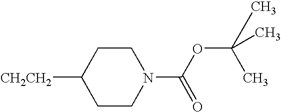 |
| 234 | H | 0 | CH═CH | H | 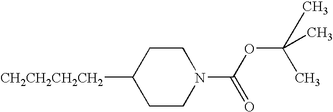 |
| 235 | H | 0 | CH═CH—CH═CH | H | 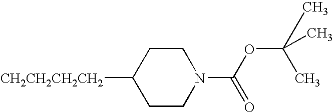 |
| 236 | H | 0 | C≡C | H | 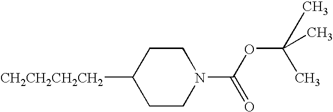 |
| 237 | H | 0 | (CH═CH)₃ | H | 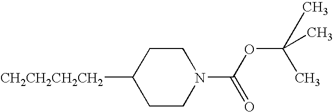 |
| 238 | H | 0 | CH═CH | H | 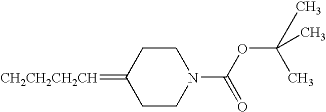 |
| 239 | H | 0 | CH═CH | H | 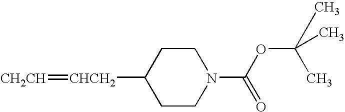 |
| 240 | H | 0 | CH═CH | H | 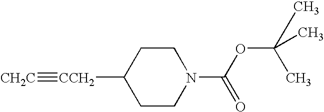 |
| 241 | H | 0 | CH═CH | H | 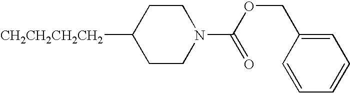 |
| 242 | H | 0 | CH═CH | H | 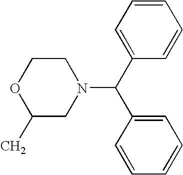 |
| 243 | H | 0 | CH═CH—CH═CH | H | 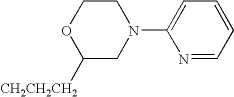 |
| 244 | H | 0 | CH═CH | H | 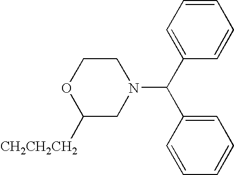 |
| 245 | H | 0 | CH═CH | H | 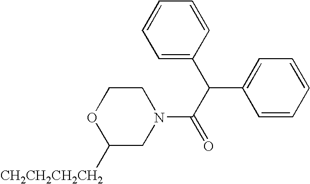 |
| 246 | H | 0 | CH═CH | H | 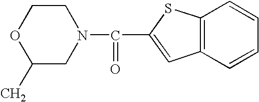 |
| 247 | H | 0 | CH═CH—CH═CH | H | 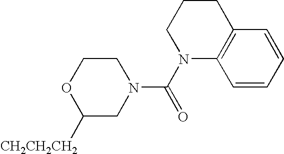 |
| 248 | H | 0 | CH═CH | H | 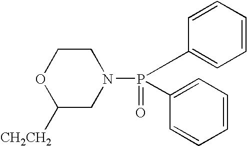 |
| 249 | H | 0 | CH═CH—CH═CH | H | 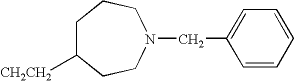 |
| 250 | H | 0 | CH═CH | H | 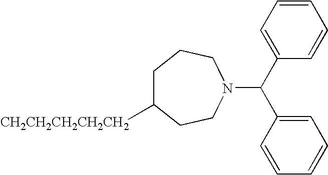 |
| 251 | H | 0 | CH═CH | H | 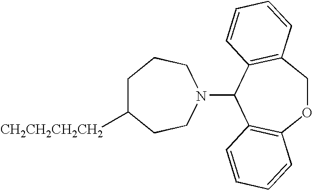 |
| 252 | H | 0 | CH═CH | H | 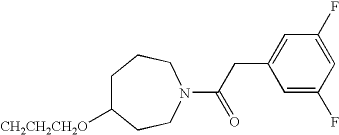 |
| 253 | H | 0 | CH═CH—CH═CH | H | 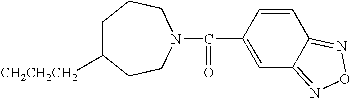 |
| 254 | H | 0 | C≡C | H | 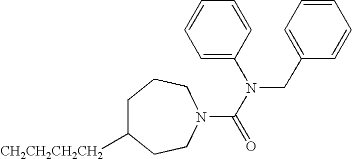 |
| 255 | H | 0 | CH═CH | H | 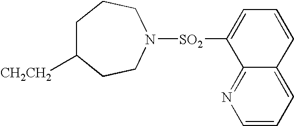 |
| 256 | H | 0 | CH═CH | H | 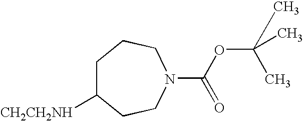 |
| 257 | H | 0 | CH═CH | H | 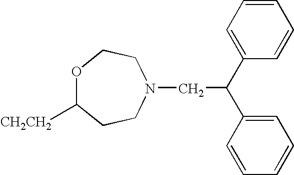 |
| 258 | H | 0 | CH═CH | H | 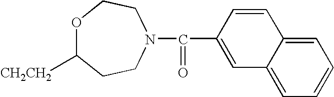 |
| 259 | H | 0 | CH═CH | H | 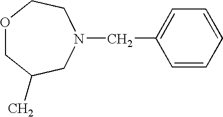 |
| 260 | H | 0 | CH═CH—CH═CH | H | 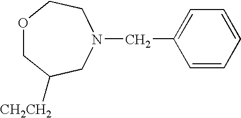 |

More preferably, the inhibitor is (E)-N-[4-(1-benzoylpiperidin-4-yl)butyl]-3-(pyridine-3-yl)-acrylamide.

Synthesis methods are for example described in EP 0 923 570.

In a preferred embodiment, the present invention concerns the use of an inhibitor as defined above for the preparation of a medicament used in the treatment of rheumatoid arthritis.

Advantageously, the present invention concerns the use of (E)-N-[4-(1-benzoylpiperidin-4-yl)butyl]-3-(pyridine-3-yl)-acrylamid for the preparation of a medicament for treating rheumatoid arthritis.

In a preferred embodiment, the present invention concerns the use of an inhibitor as defined above for the preparation of a medicament used in the treatment of endotoxemia.

Advantageously, the present invention concerns the use of (E)-N-[4-(1-benzoylpiperidin-4-yl)butyl]-3-(pyridine-3-yl)-acrylamid for the preparation of a medicament for treating endotoxemia.

Further, the present invention relates to a process to manufacture a medicament for treating inflammatory diseases wherein an effective amount of an inhibitor of the formation of nicotinamide adenyl dinucleotide is used.

In the process according to the present invention the inhibitor is preferably a competitive or noncompetitive inhibitor of the enzyme nicotinamide phosphoribosyltransferase.

Preferably, the inhibitor is a compound as defined above.

More preferably, the inhibitor is (E)-N-[4-(1-benzoylpiperidin-4-yl)butyl]-3-(pyridine-3-yl)-acrylamide.

In a first embodiment, the medicament is intended for treating rheumatoid arthritis.

In a second embodiment, the medicament is intended for treating endotoxemia.

In the process according to the present invention, the effective amount of the inhibitor may be administrated to the patient in an amount and for a time sufficient to induce a sustained amelioration of symptoms.

According to the invention, the dosage ranges of the inhibitor may vary with the administration routes, as well as the state of the patient (age, sex, body weight, extent of the disease etc.). Ideally, the dosage ranges may be between 1 mg to 100 mg/kg of body weight/day.

In the process to manufacture a medicament, a galenic composition comprising a therapeutically effective amount of an inhibitor according to the invention with at least a pharmaceutical acceptable carrier, can be prepared in a manner known per se and is suitable for enteral, such as oral or rectal, and parenteral administration to mammals, including man.

In preparing the compositions for oral dosage form, any convenient pharmaceutical media may be employed. For example, water, glycols, oils, alcohols, flavouring agents, preservatives, colouring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or non-aqueous techniques.

A tablet containing the composition of this invention may be prepared by compression or molding, optionally with one or more accessory ingredients or adjuvants.

Compressed tablets may be prepared by compressing, in a suitable machine, the combination partners in a free-flowing form such as powder or granules, optionally mixed with a binder, lubricant, inert diluents, surface active or dispersing agent. Moulded tablets may be made by moulding in a suitable machine, a mixture of the powdered compound moistened with an inert liquid diluent. Each tablet preferably contains from about 10 mg to about 2 g of the combination partners and each cachet or capsule preferably containing from about 10 mg to about 2 g of the combination partners.

Pharmaceutical compositions suitable for parenteral administration may be prepared as solutions or suspensions of the combination partners in water. A suitable surfactant can be included such as, for example, hydroxypropylcellulose. Dispersions can also be prepared in glycerol, liquid polyethylene glycols, propylene glycols, and mixtures thereof in oils. Further, a preservative can be included to prevent the detrimental growth of micro-organisms.

Pharmaceutical compositions suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy use with a syringe. The pharmaceutical compositions must be stable under the conditions of manufacture and storage; thus, preferably should be preserved against the contaminating action of micro-organisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

The present invention also pertains to a pharmaceutical kit comprising at least an effective amount of an inhibitor of the formation of nicotinamide adenyl dinucleotide together with printed instructions for use in the treatment of inflammatory diseases.

In the pharmaceutical kit according to the present invention, the inhibitor of the formation of nicotinamide adenyl dinucleotide is preferably a compound as described above.

In the pharmaceutical kit according to the present invention, the inhibitor of the formation of nicotinamide adenyl dinucleotide is preferably (E)-N-[4-(1-benzoylpiperidin-4-yl)butyl]-3-(pyridine-3-yl)-acrylamide.

In a first embodiment, the pharmaceutical kit of the present invention comprises printed instructions for use in the treatment of rheumatoid arthritis.

In a second embodiment, the pharmaceutical kit of the present invention comprises printed instructions for use in the treatment of endotoxemia.

The pharmaceutical kit according to the present invention may comprise a container comprising at lcast said inhibitor. In a preferred embodiment, the kit container may further include a pharmaceutically acceptable carrier. The kit may further include a sterile diluent, which is preferably stored in a separate additional container.

Finally, the present invention concerns a method of treating inflammatory diseases comprising administering to a subject an effective amount of an inhibitor of the formation of nicotinamide adenyl dinucleotide.

Preferably, the invention concerns a method of treating inflammatory diseases wherein the inhibitor is a noncompetitive or competitive inhibitor of the enzyme nicotinamide phosphoribosyltransferase.

More preferably, the invention concerns a method of treating inflammatory diseases wherein the inhibitor is a compound as defined above.

In a first aspect, the invention concerns methods for treating rheumatoid arthritis.

In a second aspect, the invention concerns methods for treating endotoxemia.

This invention will be better understood from the Experimental Details that follow. However, one skilled in the art will readily appreciate that the specific methods and results discussed are merely illustrative of the invention as described more fully in the claims which follow thereafter, and are not to be considered in any way limited thereto.

### FIGURES LEGEND

FIG. 1: Direct relationship between NAD levels in cells and pro-inflammatory cytokine secretion.

Human monocytic cell line THP-1 was cultured overnight in the presence of graded doses of nicotinic acid (NA), a precursor of NAD, and then stimulated with lipopolysaccharide (LPS) from gram-negative bacteria for 2 h. The cell supernatant was tested for tumor necrosis factor (TNF) content by ELISA and the intracellular pool of NAD was measured by an enzymatic assay.

FIG. 2: FK866, a competitive inhibitor of NMPRT, inhibits proinflammatory cytokine production in inflammatory cells in response to LPS.

Human monocytic cell line THP-1, human peripheral blood mononuclear cells (PBMC), human monocyte-derived dendritic cells, or mouse peritoneal macrophages were isolated and cultured overnight with increasing doses of FK866, and then stimulated with LPS for 6 h. The culture supernatants were tested for TNF and IL-6 content by ELISA

FIG. 3: FK866 inhibits the secretion of TNFα at a posttranscriptional level.

The murine RAW264.7 cell line was stimulated with LPS in the presence of FK866. TNF protein concentrations were determined by ELISA (above panel) while mRNA levels were determined by RT-PCR (below panel).

FIG. 4: FK866 inhibits the secretion of pro-inflammatory cytokines TNFα, IL-12 and IL-23 in human dendritic cells.

Human dendritic cells (mean and SD from 5 different donors) were incubated with 20 nM FK866 before stimulation with LPS or LPS+IFNγ. Pro-inflammatory cytokine levels in the supernatant were measured by ELISA after 16 h of culture.

FIG. 5: Correlation between proinflammatory cytokine secretion and NAD levels in the cell.

(A) The mouse macrophage cell line RAW264.7 was cultured overnight with increasing doses of FK866, and then stimulated with LPS for 2 h. The culture supernatants were tested for TNF content by ELISA and intracellular NAD levels were measured by an enzymatic assay.

(B) RAW264.7 cells were cultured overnight in the presence of FK866 and the intracellular pool of NAD was restored by co-incubation of the cells with nicotinic acid (NA). Cells were then stimulated with LPS for 2 h and the culture supernatant was tested for TNF content by ELISA.

(C) RAW264.7 cells were cultured overnight in the presence of FK866 and NAD levels were maintained by culturing the cells in the presence of extracellular NAD. Cells were then stimulated with LPS for 2 h and the culture supernatant was tested for TNF content by ELISA.

FIG. 6: Inhibition of proinflammatory cytokine secretion induced by lowering intracellular NAD levels is not due to apoptosis induction.

(A) Human PBMC were isolated and cultured overnight with increasing doses of FK866, and then stimulated with LPS for 6 h. At the end of the culture, cell viability was assessed using the MTT (3-(4,5-Dimethylthiazol-2-yl)-2,5-diphenyltetrazolium bromide) assay.

(B) Human monocyte-derived dendritic cells were isolated and cultured overnight with increasing doses of FK866, and then stimulated with LPS for 6 h. At the end of the culture, the survival of cells was measured by Annexin-V and propidium iodide staining.

FIG. 7: Nicotinamide mononucleotide (NMN) reverts the inhibitory effects of FK866 on intracellular levels and TNFα secretion.

THP-1 cells were incubated in the presence of 10 nM FK866 and graded doses of NMN. Cells were further stimulated with LPS and intracellular NAD levels and TNFα secretion were determined using standard assays.

FIG. 8: Reduction of disease severity of rheumatoid arthritis (RA) in an experimental mouse model of collagen-induced RA after treatment with FK866.

Male DBA/1 mice between 8-10 weeks of were immunized intradermally at the base of tail with 100 μg of native type II collagen (CII), emulsified in complete Freund's adjuvant containing 5 mg/ml mycobacterium tuberculosis. Twenty-one days later, the mice were boosted with 100 μg collagen in incomplete Freund's adjuvant intradermally at the base of the tail. From day 15 after the first immunization onward, mice are examined daily for the onset of clinical arthritis. The severity of arthritis is scored on a 3-point scale, where 0=normal appearance, 1=mild swelling and/or erythema, 2=pronounced swelling and erythema, and 3=joint rigidity. Each limb is graded, resulting in a maximal clinical score of 12 per animal. Treatment with FK866 was administered twice daily at 10 mg/kg intraperitoneally for a total of 15 days from the day when CIA became clinically detectable (clinical scoring≧1). Values are ± s.e.m. of clinical score with 10 animals per group.

FIG. 9: Induction of NAMPT expression in collagen-induced arthritis.

Sera (a) and tissue extracts of paws (b) from CIA at day 14 (n=8) and from non-arthritic, non-immunized, naïve (n=7) mice were prepared and analyzed by NAMPT ELISA. *P<0.05 arthritic versus naïve in panel a and b.

FIG. 10: Clinical, histological and biochemical effects of NAMPT inhibition on established arthritis.

(a) Dose-response effect of FK866: test mice were treated twice daily ip with FK866 2, 5, or 10 mg/kg (n=10 in each group) during 15 days. Placebo mice received vehicle only (n=10). (b) Severity of arthritis in CIA mice receiving FK866 10 mg/kg ip twice daily or etanercept 15 mg/kg every three days (n=10 in each group) over 15 days. Mice groups were compared by two-way ANOVA. *P<0.05 FK866 or etanercept versus placebo in panel a and b.

Test mice (n=20) were twice daily treated ip with 10 mg/kg of FK866 from the first day onward of appearance of clinical arthritis (clinical score>1) during 14 days. Placebo mice (n=20) received vehicle only. Groups of animals were compared with respect to variation of their clinical scoring, and of their weight (d) by statistical analysis using the two-way ANOVA. A semi-quantitative histological evaluation was performed on the knee sections using a 4 points (0-3) scoring system to evaluate inflammatory infiltrate and synovial hyperplasia. (c) Circulating SAA levels: Sera from placebo- and FK866-treated CIA mice at day 14 (n=8 and n=7, respectively) were prepared and analyzed by SAA ELISA according to the manufacturer's instructions.

FIG. 11: FK866 reduces intracellular NAD in inflammatory cells in vivo.

Mice were treated with thioglycollate to elicit PEC, and then received 10 mg/kg FK866 by ip injection. PEC were obtained by lavage after different time points and intracellular NAD was determined. Data are mean±sem of 3 mice per group.

FIG. 12: FK866 inhibits TNFα production after LPS challenge.

Mice were treated with thioglycollate to elicit PEC, and then received 10 mg/kg FK866 or placebo by ip injection 7 h before ip challenge with LPS. Mean serum TNFα at 90 min+sem of 3 mice per group is shown. PEC were obtained by lavage and intracellular NAD was determined. Data are mean±sem of 3 mice per group.

### EXAMPLES

### Example 1

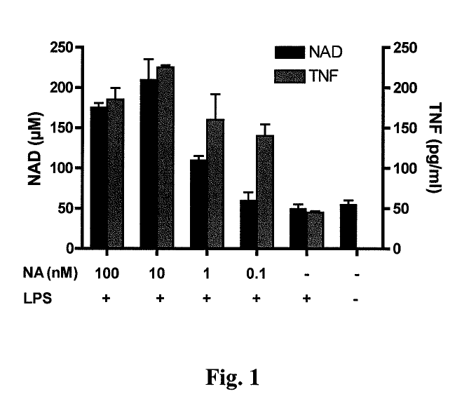

This example illustrates the direct relationship between NAD levels in cells and pro-inflammatory cytokine secretion. Human monocytic cell line THP-1 was cultured overnight in the presence of nicotinic acid, a precursor of NAD, and then stimulated with lipopolysaccharide (LPS) from gram-negative bacteria for 2 h. The cell supernatant was tested for tumor necrosis factor (TNF) content by ELISA and the intracellular pool of NAD was measured by an enzymatic assay. FIG. 1 shows that TNF secretion and NAD levels were increased in parallel in a dose-dependent fashion in the presence of nicotinic acid.

### Example 2

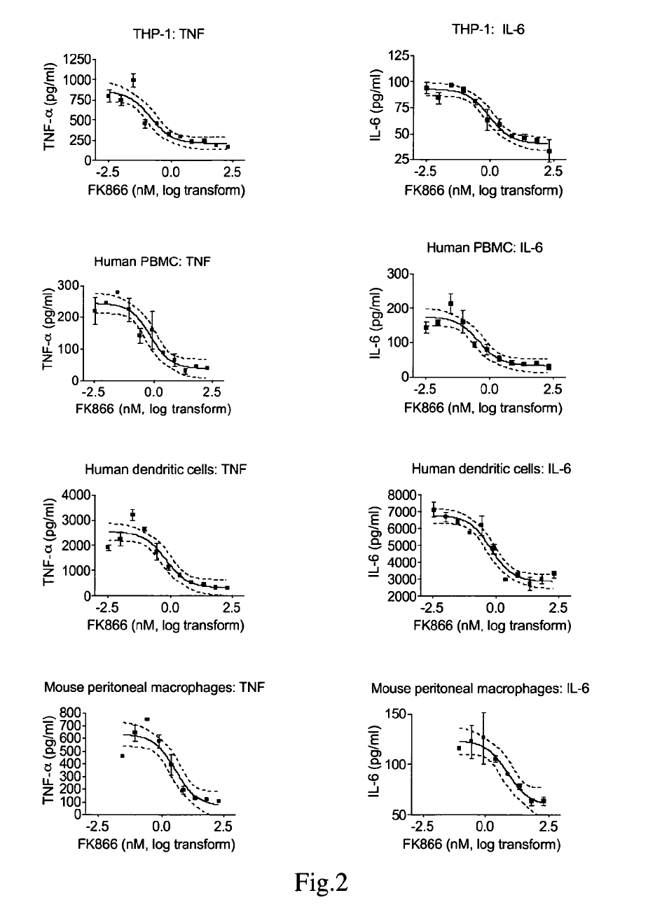

This example describes the inhibition of pro-inflammatory cytokines production by the competitive small molecular weight compound inhibitor of NMPRT, ((E)-N-[4-(1-benzoylpiperidin-4-yl)butyl]-3-(pyridin-3-yl)acrylamide, designated FK866). Human monocytic cell line THP-1, human peripheral blood mononuclear cells (PBMC), human monocyte-derived dendritic cells, or mouse peritoneal macrophages were isolated and cultured overnight with increasing doses of FK866, and then stimulated with LPS for 6 h. The culture supernatants were tested for TNF and IL-6 content by ELISA. FIG. 2 shows that FK866 inhibited cytokine secretion in a dose-dependent fashion in all inflammatory cells tested.

### Example 3

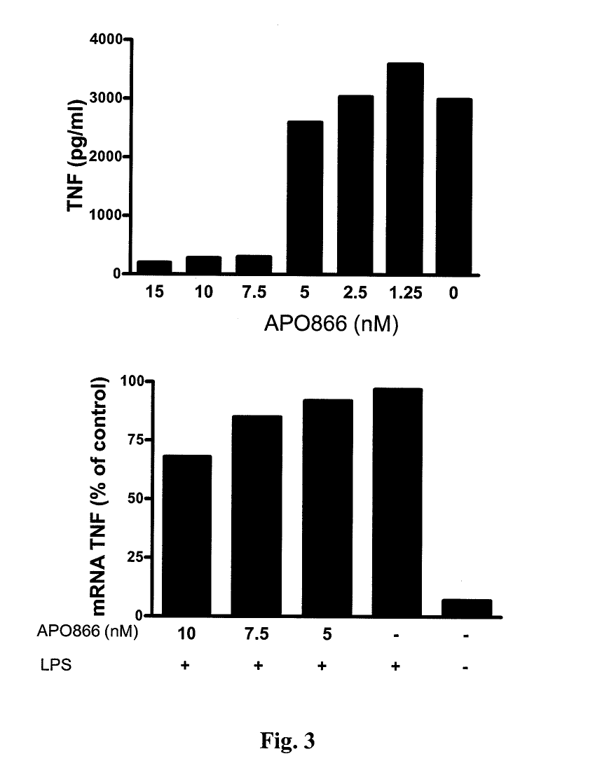

This example illustrates that FK866 inhibits TNFα secretion at a posttranscriptional level. The murine RAW 264.7 cell line was incubated in the presence of graded doses of FK866, stimulated with microbial products, and levels of TNFα released in the supernatant evaluated by ELISA. As shown in FIG. 3, FK866 strongly inhibited TNFα secretion in this experimental setting. FK866 did not significantly affect TNFα mRNA levels, showing that intracellular NAD regulates the translational efficiency of TNFα mRNA.

### Example 4

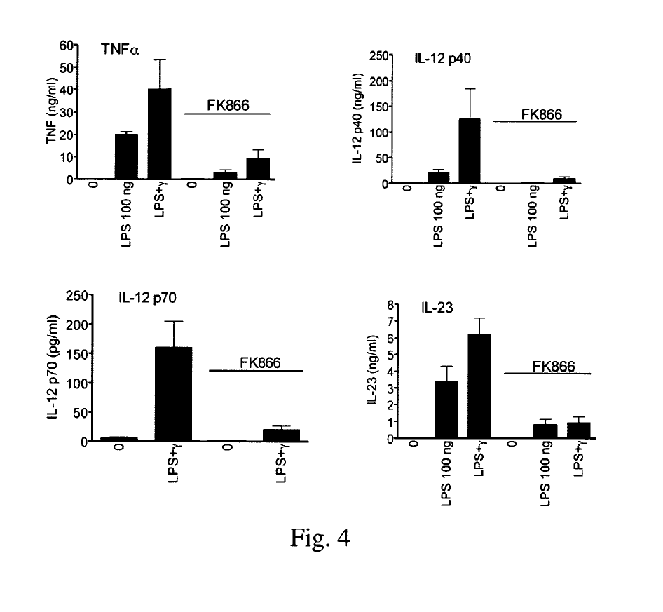

This example describes the inhibition of other pro-inflammatory cytokine production, in addition to TNFα, IL-1β and IL-6, by FK866. Human dendritic cells from 5 different donors were cultured with FK866 before stimulation with LPS or LPS+IFNγ. The culture supernatants were tested for TNFα, IL-12 (p40 and p70), and IL-23 content by ELISA. FIG. 4 shows that FK866 inhibited TNFα (as described in our application) as well as IL-12 and IL-23 production.

### Example 5

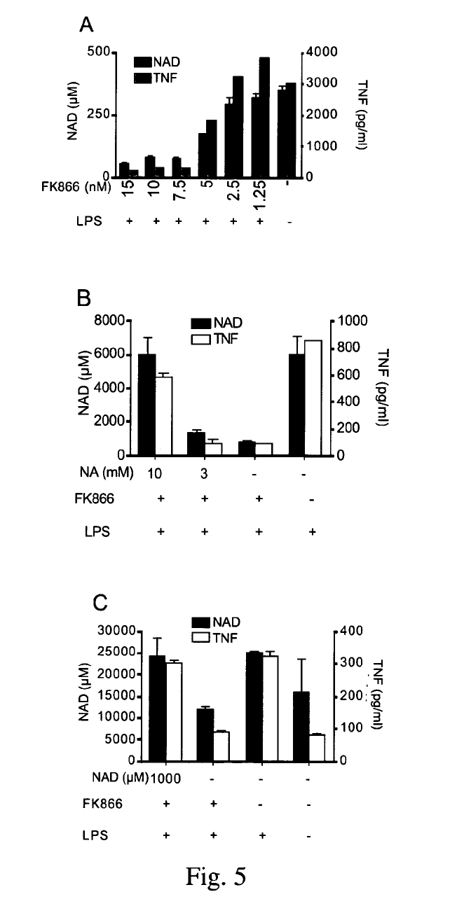

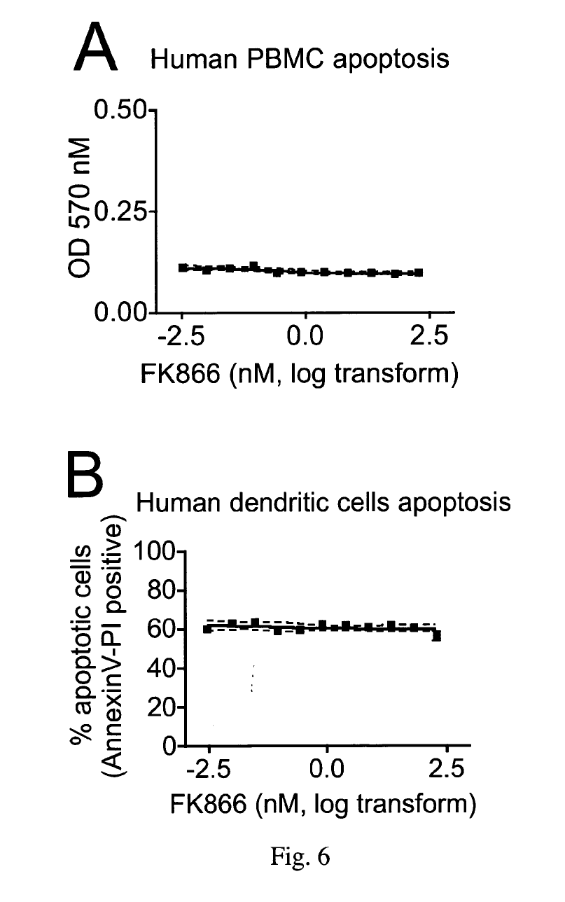

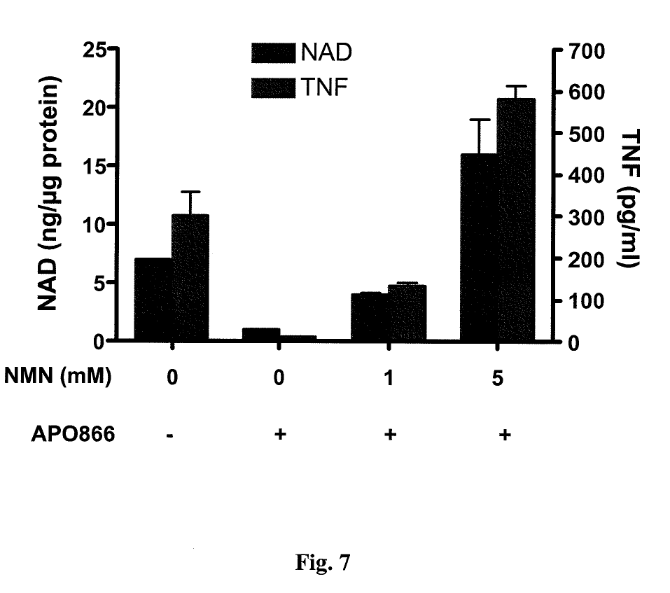

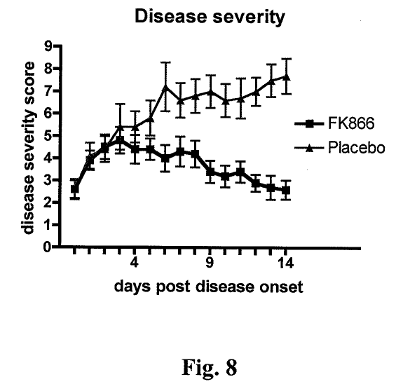

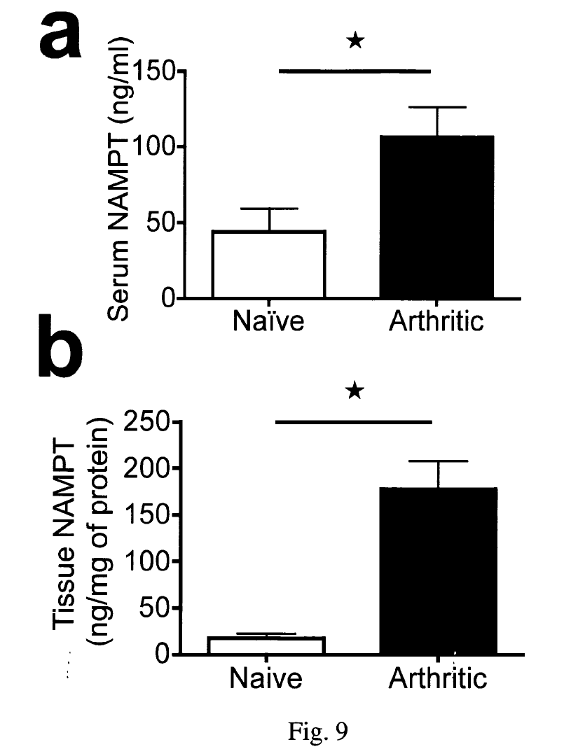

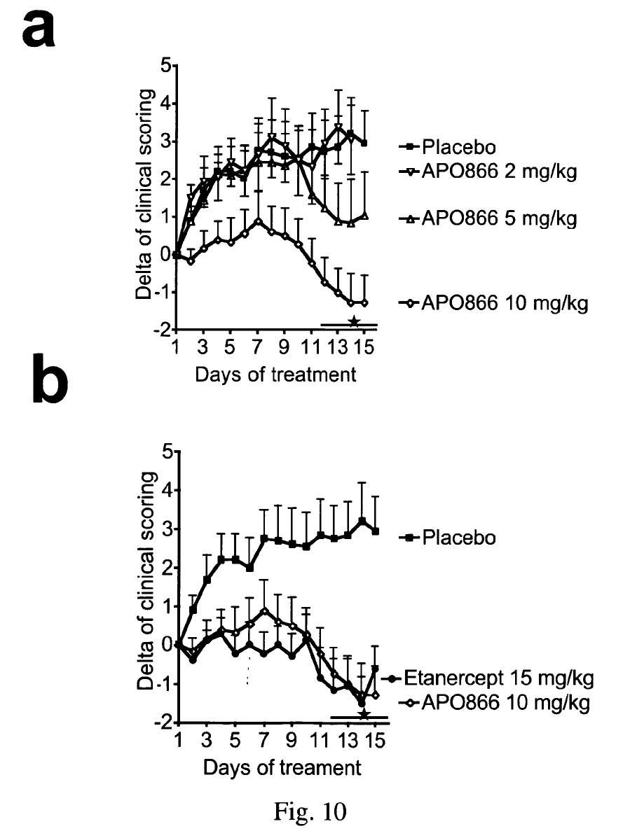

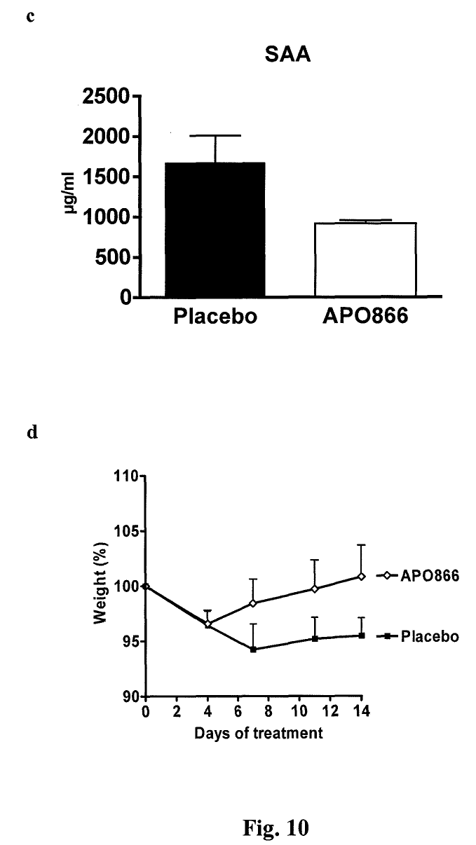

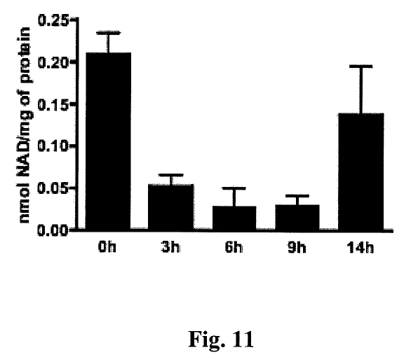

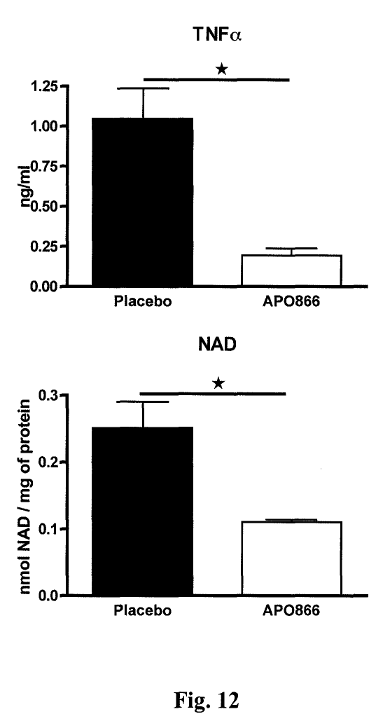

This example illustrates the correlation between proinflammatory cytokine secretion and NAD levels in the cell. The mouse macrophage cell line RAW264.7 was cultured overnight with increasing doses of FK866, and then stimulated with LPS for 2 h. The culture supernatants were tested for TNF content by ELISA and intracellular NAD levels were measured by an enzymatic assay. FIG. 5A shows that inhibition of TNF production correlated with the inhibition of intracellular NAD levels.

In another experiment, RAW264.7 cells were cultured overnight in the presence of FK866 and the intracellular pool of NAD was restored by co-incubation of the cells with nicotinic acid. Nicotinic acid is a precursor of NAD, but its transformation into NAD is not dependent upon NMPRT and is thus not inhibited by FK866. Cells were then stimulated with LPS for 2 h and the culture supernatant was tested for TNF content by ELISA. FIG. 5B shows that addition of nicotinic acid maintained high levels of NAD, even in the presence of FK866. The synthesis of TNF was also restored showing the direct relationship between NAD levels in cells and pro-inflammatory cytokine secretion.

In another experiment, RAW264.7 cells were cultured overnight in the presence of FK866 and NAD levels were maintained by culturing the cells in the presence: of extra-cellular NAD. Cells were then stimulated with LPS for 2 h and the culture supernatant was tested for TNF content by ELISA. FIG. 5C shows that NAD levels remained high even in the presence of FK866, and TNF synthesis was restored.

### Example 6

This example illustrates that the inhibition of proinflammatory cytokine secretion induced by lowering intracellular NAD levels is not due to apoptosis induction. Human PBMC were isolated and cultured overnight with increasing doses of FK866, and then stimulated with LPS for 6 h. At the end of the culture, cell viability was assessed using the MTT (3-(4,5-Dimethylthiazol-2-yl)-2,5-diphenyltetrazolium bromide) assay. FIG. 6A shows that cell viability was not affected by FK866.

In another experiment, human monocyte-derived dendritic cells were isolated and cultured overnight with increasing doses of FK866, and then stimulated with LPS for 6 h. At the end of the culture, the survival of cells was measured by Annexin-V and propidium iodide staining and no difference in survival was observed in cells treated with FK866 compared to untreated cells (FIG. 6B). This shows that inhibition of NAD and pro-inflammatory cytokine secretion was not simply due to cell death induction.

### Example 7

This example illustrates that NAMPT is the only molecular target of FK866. The human THP-1 monocytic cell line was cultured in the presence of medium or FK866 and stimulated by LPS. Addition of nicotinamide mononucleotide (NMN), the product of the NAMPT-catalyzed reaction, restored both intracellular NAD levels and TNFα production, despite continuous exposure to FK866 (FIG. 7). The underlying mechanism by which NMN exerts its effect is linked to NAD generation via the conversion of NMN to NAD, catalyzed by NMNAT. These results confirm both the specificity of action of FK866 and the role of intracellular NAD in regulating TNFα secretion.

### Example 8

This example describes the reduction of disease severity of rheumatoid arthritis (RA) in an experimental mouse model of collagen-induced RA after treatment with FK866. RA is an autoimmune disorder characterized by chronic inflammation of the joints leading to their destruction. Pro-inflammatory cytokines play a major role in the development and maintenance of the disease, and blocking of TNF or IL-1 for alleviating symptoms of RA is now well established in clinical practice. Male DBA/1 mice between 8-10 weeks of were immunized intradermally at the base of tail with 100 μg of native type II collagen (CII), emulsified in complete Freund's adjuvant containing 5 mg/ml mycobacterium tuberculosis. Twenty-one days later, the mice were boosted with 100 μg collagen in incomplete Freund's adjuvant intradermally at the base of the tail. From day 15 after the first immunization onward, mice are examined daily for the onset of clinical arthritis. The severity of arthritis is scored on a 3-point scale, where 0=normal appearance, 1=mild swelling and/or erythema, 2=pronounced swelling and erythema, and 3=joint rigidity. Each limb is graded, resulting in a maximal clinical score of 12 per animal. Treatment with FK866 was administered twice daily at 10 mg/kg intraperitoneally for a total of 15 days from the day when CIA became clinically detectable (clinical scoring≧1). Results shown in FIG. 8 indicate that FK866 ameliorates the symptoms of arthritis in an animal model of RA.

### Example 9

This example describes that FK866-targeted enzyme NAMPT expression is upregulated in collagen-induced arthritis (CIA). During CIA, the level of NAMPT was significantly elevated in sera and paw tissue extracts from arthritic mice compared to non-arthritic naïve controls as measured by ELISA (FIGS. 9a and b, respectively). These results were also supported by NAMPT immunohistochemistry. Indeed, we found massive staining of arthritic paw and knee joints from CIA, but markedly reduced staining in non-arthritic joints or joints from naïve mice. In affected joints, NAMPT staining was prominent in synoviocytes of the synovial lining layer (SLL), sub-intimal synovium and pannus (P) and in some inflammatory cells. Most of the blood vessels were also positive. In addition, some positive chondrocytes were observed in both normal and arthritic joints.

### Example 10

This example illustrates that NAMPT inhibition with FK866 reduces established collagen-induced arthritis. FK866 was administered from the day following the appearance of the first clinical symptoms of arthritis, and continued for 15 days. FK866 had a marked protective dose-dependent effect on CIA, with a maximal therapeutic effect when administered at 10 mg/kg (FIG. 10a). The beneficial effect was apparent within 10 days following the commencement of treatment, and with an activity similar to etanercept (anti-TNFα treatment) (FIG. 10b). To gain more insight into the inhibitory mechanism of action of FK866 on CIA, we repeated the CIA curative experiment using the optimal dose of FK866 and analyzed more parameters. Paws from FK866-treated mice showed minimal signs of inflammation after 2 weeks of treatment whereas paws from placebo-treated mice were still inflamed, and this was also reflected in the clinical scoring. Additionally, these in vivo clinical observations were consistent with histology of knees and paws, where much less inflammation was observed in the FK866-treated group. Knee joints of placebo mice and mice treated with FK866 were assessed for inflammatory infiltrate and synovial hyperplasia. Histological sections revealed a statistically significant decrease in inflammatory infiltrate and hyperplasia in mice treated with FK866 as compared to placebo-treated controls. Serum amyloid A protein (SAA) levels, which reflect the systemic inflammatory response, were decreased in FK866-treated mice (FIG. 10c), further suggesting the anti-inflammatory effect of FK866 administration. Amongst the potential molecular mechanisms involved in the amelioration of CIA by FK866 is the reduction of pro-inflammatory cytokines. Expression of different cytokines was investigated in paw tissue extracts at the end of the experiment. TNFα was below the level of detection of the assay Locally produced IL-1β and IL-6 were significantly reduced in FK866-treated animals. MCP-1 was decreased, and finally IL-10 secretion remained unchanged by FK866 treatment.

We observed no signs of toxicity resulting from the treatment with FK866 since the weight of the mice was comparable between placebo- and FK866-treated groups (FIG. 10d). Indeed, FK866 was well tolerated, no premature death occurred in the treated group and the corresponding histopathology of liver, spleen, lung, gut, kidney, inguinal lymph nodes and brain in this group was no different from control animals. In addition, liver toxicity was also ruled out as similar alanine aminotransferase low levels were measured in FK866-treated versus control mice. Finally, hematological examination showed similarity between the treated and control mice (Table 1).

TABLE 1

| | |
|---|---|
| PLACEBO | APO866 |

| | mean | SD | mean | SD |
|---|---|---|---|---|
| RBC (10³/mm³) | 13.6 | 4.9 | 9.4 | 1.4 |
| WBC (10⁶/mm³) | 12.1 | 0.4 | 11.5 | 0.7 |
| HGB (g/dl) | 14.8 | 0.6 | 14.1 | 0.8 |
| HCT (%) | 55 | 2.4 | 52.8 | 2.6 |
| PLT (10³/mm³) | 1832.6 | 77 | 1688 | 181 |
| % LYMPHO | 54.6 | 7.6 | 60 | 7.4 |
| % MONO | 12.1 | 2.1 | 10.4 | 1.3 |
| % NEUTRO | 33.3 | 6.7 | 29.7 | 6.5 |

To verify that FK866-treated mice generated an adequate immune response against type II collagen, total anti-collagen IgG levels were measured by ELISA at the end of the therapy (day 15). No significant difference was observed in anti-collagen IgG levels between control and FK866-treated mice (control mice: 140+/−20.2 arbitrary units (n=18), treated mice: 106.5+/−17.6 arbitrary units (n=15)). Collectively, these data show that the beneficial effects of FK866 on established CIA were neither due to toxicity nor to impaired immune response to collagen II, but to an impaired secretion of inflammatory cytokines.

### Example 11

This example illustrates that FK866 reduces intracellular NAD level in inflammatory cells in vivo. Naïve mice were treated ip with thioglycollate to elicit inflammatory cells, and then FK866 was administered ip at 10 mg/kg. Peritoneal exudates cells (PEC) were obtained by lavage at different time points after treatment, and intracellular NAD levels were determined using an enzymatic assay. FIG. 11 shows that FK866 induced a significant time-dependent NAD depletion in macrophages in vivo with a nadir at 9 h and recovery around 14 h after injection.

### Example 12

This example illustrates that NAMPT inhibition reduces circulating TNFα during endotoxemia and correlates with diminished intracellular NAD in inflammatory cells. Naïve mice were treated ip with thioglycollate to elicit inflammatory cells, and then were treated ip with placebo or 10 mg/kg FK866 7 h before an intraperitoneal injection of LPS. Mice were bled 90 min later for evaluation of serum TNFα levels. As shown in FIG. 12, FK866 induced a significant decrease in circulating TNFα levels compared to placebo. This decrease in TNFα secretion was accompanied by a significant decrease in intracellular NAD in PECs obtained from the same mice.

1. Method of treating inflammatory diseases comprising administering to a subject an effective amount of an inhibitor of the formation of nicotinamide adenyl dinucleotide.

2. Method according to claim 1, wherein the inhibitor is a noncompetitive or competitive inhibitor of the enzyme nicotinamide phosphoribosyltransferase.

3. Method according to claim 1, wherein the inhibitor is a compound according to formula (I): 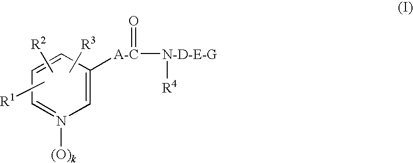
wherein
R¹ is selected from the group consisting of hydrogen, halogen, cyano, C₁-C₆-alkyl, trifluoromethyl, C₃-C₈-cycloalkyl, C₁-C₄-hydroxyalkyl, hydroxy, C₁-C₄-alkoxy, benzyloxy, C₁-C₄-alkanoyloxy, C₁-C₄-alkylthio, C₂-C₅-alkoxycarbonyl, aminocarbonyl, C₃-C₈-dialkylaminocarbonyl, carboxy, phenyl, phenoxy, pyridyloxy, and NR⁵R⁶, wherein
R⁵ and R⁶ are selected independently from each other from hydrogen and C₁-C₆-alkyl,
R² is selected from hydrogen, halogen, C₁-C₆-alkyl, trifluoromethyl and hydroxy, wherein
R¹ and R², in the case they are adjacent, optionally form a bridge which is selected from the group of bridge members —(CH₂)₄— and —(CH═CH)₂— and —CH₂O—CR⁷R⁸—O—, wherein
R⁷ and R⁸ are independent from each other, hydrogen or C₁-C₆-alkyl,
R³ is selected from hydrogen, halogen and C₁-C₆-alkyl,
R⁴ is selected from hydrogen, C₁-C₆-alkyl, C₃-C₆-alkenyl, hydroxy, C₁-C₆-alkoxy and benzyloxy,
k is 0 or 1,
A is selected from
C₂-C₆-alkenylene, which is optionally substituted one to three-fold by C₁-C₃-alkyl, hydroxy, fluorine, cyano, or phenyl,
C₄-C₆-alkadienylene, which is optionally substituted once or twice by C₁-C₃-alkyl, fluorine, cyano, or phenyl,
1,3,5-hexatrienylene, which is optionally substituted by C₁-C₃-alkyl, fluorine, or cyano, and ethinylene,
D is selected from
C₁-C₁₀-alkylene, optionally substituted once or twice by C₁-C₃-alkyl or hydroxy,
C₂-C₁₀-alkenylene, optionally substituted once or twice by C₁-C₃-alkyl or hydroxy, wherein the double bond optionally is to ring E.
C₃-C₁₀-alkinylene, optionally substituted once or twice by C₁-C₃-alkyl or hydroxy, and
the group consisting of C₁-C₁₀-alkylene, C₂-C₁₀-alkenylene and C₃-C₁₀-alkinylene, in which one to three methylene units are isosterically replaced by O, S, NR⁹, CO, SO or SO₂, wherein
R⁹ is selected from hydrogen, C₁-C₃-alkyl, C₁-C₆-acyl and methanesulfonyl,
E is selected from 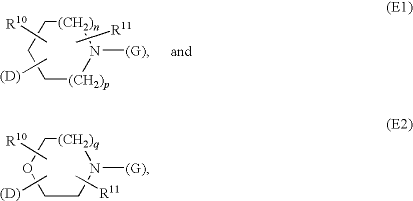
wherein the heterocyclic ring optionally has a double bond and
n and p are, independent of each other, 0, 1, 2 or 3, with the proviso that n+p≦4,
q is 1 or 2,
R¹⁰ is selected from hydrogen, C₁-C₃-alkyl, hydroxy, and hydroxymethyl, carboxy and C₂-C₇-alkoxycarbonyl,
R¹¹ xis hydrogen or an oxo group adjacent to the nitrogen atom,
G is selected from hydrogen,
G1, G2, G3, G4 and G5, wherein
G1 represents the residue
—(CH₂)ᵣ—(CR¹³R¹⁴)ₛ—R¹² (G1)
wherein
r is 0, 1 or 2 and
s is 0 or 1,
R¹² is selected from hydrogen, C₁-C₆-alkyl, C₃-C₆-alkenyl, C₃-C₆-alkinyl, C₃-C₈-cycloalkyl, benzyl, phenyl,
the group consisting of monocyclic aromatic five- and six-membered heterocycles, which contain one to three hetero-atoms selected from N, S and O and are either bound directly or over a methylene group,
the group consisting of anellated bi- and tricyclic aromatic or partially hydrogenated carbocyclic ring systems with 8 to 16 ring atoms and at least one aromatic ring, wherein the bond occurs either over an aromatic or a hydrogenated ring and either directly or over a methylene group, and
the group consisting of anellated bi- and tricyclic aromatic or partially hydrogenated heterocyclic ring systems with 8 to 16 ring atoms and at least one aromatic ring, wherein one to three ring atoms are selected from N, S and O and the bond occurs either over an aromatic or a hydrogenated ring, and either directly or over a methylene group,
R¹³ has the same meaning as R¹², but is selected independently thereof,
R¹⁴ is selected from hydrogen, hydroxy, methyl, benzyl, phenyl,
the group consisting of monocyclic aromatic five- and six-membered heterocycles, which contain one to three hetero-atoms selected from N, S and O and are bound either directly or over a methylene group,
the group consisting of anellated bi- and tricyclic aromatic or partially hydrogenated carbocyclic ring systems with 8 to 16 ring atoms and at least one aromatic ring, wherein the bond occurs either over an aromatic or a hydrogenated ring and either directly or over a methylene group, and
the group consisting of anellated bi- and tricyclic aromatic or partially hydrogenated heterocyclic ring systems with 8 to 16 ring atoms and at least one aromatic ring, wherein one to three ring atoms are selected from N, S and O and the bond occurs either over an aromatic or a hydrogenated ring and either directly or over a methylene group,
G2 is selected from the residues 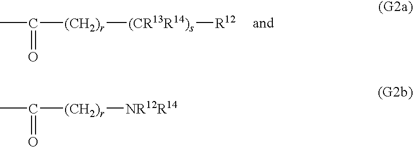
wherein the substituents R¹² and R¹⁴ have the above meaning, or the group
—NR¹²R¹⁴
is a nitrogen-containing heterocycle bound over the nitrogen atom, the nitrogen-containing heterocycle being selected from
the group consisting of saturated and unsaturated monocyclic, four- to eight-membered heterocycles, which, aside from the essential nitrogen atom,
optionally contain one or two further hetero-atoms selected from N, S and O, and the group consisting of saturated and unsaturated bi- or tricyclic, anellated or bridged heterocycles with 8 to 16 ring atoms, which, aside from the essential nitrogen atom, optionally contain one or two further hetero-atoms selected from N, S and O,
G3 is the residue
—SO₂—(CH₂)ᵣR¹² (G5)
G4 is the residue 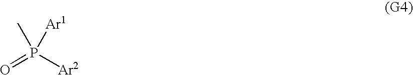
wherein
Ar¹ and Ar² are selected independently of each other from phenyl, pyridyl and naphthyl,
G5 is the residue
—COR¹⁵ (G5)
wherein
R¹⁵ is selected from trifluoromethyl, C₁-C₆-alkoxy, C₃-C₆-alkenyloxy and benzyloxy,
wherein aromatic ring systems in the substituents R¹, R², R⁴, R¹², R¹³, R¹⁴, R¹⁵, Ar¹ and Ar² and in the ring system —NR¹²R¹⁴ optionally carry independently of each other one to three substituents which are independently selected from the group consisting of halogen, cyano, C₁-C₆-alkyl, trifluoromethyl, C₃-C₈-cycloalkyl, phenyl, benzyl, hydroxy,
C₁-C₆-alkoxy, which is optionally entirely or partially substituted by fluorine, benzyloxy, phenoxy, mercapto, C₁-C₆-alkylthio, carboxy, C₁-C₆-alkoxycarbonyl, benzyloxycarbonyl, nitro, amino, mono-C₁-C₆-alkylamino, and di-(C₁-C₆-alkyl)-amino,
wherein two adjacent groups of the aromatic ring or ring system optionally form an additional ring over a methylenedioxy bridge,
tautomeres in the case of substitution of the heterocycle or in an anellated ring system by free hydroxy, mercapto and/or amino groups,
stereoisomers and/or mixtures thereof and pharmacologically acceptable acid addition salts with the exception of (E)-3-(3-pyridyl)-N-[2-(1-benzylpiperidin-4-yl)ethyl]-2-propenamide hydrochloride.

4. Method according to claim 3, wherein:
R¹ is selected from hydrogen, halogen, cyano, methyl, trifluoromethyl, hydroxy, C₁-C₄-alkoxy, ethylthio, methoxycarbonyl, tert-butoxycarbonyl, aminocarbonyl, carboxy, and phenoxy,
R² is selected from hydrogen, halogen, trifluoromethyl and hydroxy,
R³ is hydrogen or halogen,
R⁴ is selected from hydrogen, C₁-C₃-alkyl, hydroxy and C₁-C₃-alkoxy,
k is 0 or 1,
A is selected from C₂-C₆-alkenylene, optionally substituted once or twice by C₁-C₃-alkyl, hydroxy or fluorine,
C₄-C₆-alkadienylene, optionally substituted by C₁-C₃-alkyl or by 1 or 2 fluorine atoms, and 1,3,5-hexatrienylene, optionally substituted by fluorine,
D is selected from C₁-C₈-alkylene, optionally substituted once or twice by methyl or hydroxyl,
C₂-C₈-alkenylene, optionally substituted once or twice by methyl or hydroxy, wherein the double bond optionally is to ring E,
C₃-C₈-alkinylene optionally substituted once or twice by methyl or hydroxy, and the group consisting of C₁-C₈-alkylene, C₂-C₈-alkenylene and C₃-C₈-alkinylene in which one to three methylene units are isosterically replaced by O, S, NH, N(CH₃), N(COCH₃), N(SO₂CH₃) CO, SO or SO₂,
E is selected from 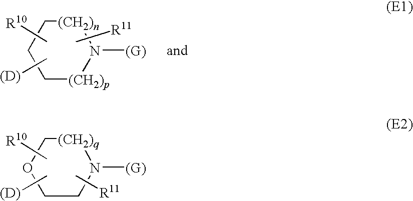
wherein the heterocyclic ring optionally has a double bond and
n and p are, independent of each other, 0, 1, 2 or 3, with the proviso that n+p≦3,
q is 1 or 2,
R¹⁰ is selected from hydrogen, C₁-C₃-alkyl, hydroxy, and hydroxymethyl,
R¹¹ is hydrogen or an oxo group which is adjacent to the nitrogen atom,
G is selected from hydrogen,
G1, G2, G3, G4 and G5, wherein
G1 represents the residue
—(CH₂)ᵣ—(CR¹³R¹⁴)ₛ—R¹² (G1)
wherein
r is 0, 1 or 2 and
s is 0 or 1,
R¹² is selected from hydrogen, C₁-C₆-alkyl, C₃-C₈-cycloalkyl, benzyl, phenyl,
the group consisting of benzocyclobutyl, indanyl, indenyl, oxoindanyl, naphthyl, dihydronaphthyl, tetrahydronaphthyl, oxotetrahydronaphthyl, biphenylenyl, fluorenyl, oxofluorenyl, anthryl, dihydroanthryl, oxodihydroanthryl, dioxodihydroanthryl, phenanthryl, dihydrophenanthryl, oxodihydrophenanthryl, dibenzocycloheptenyl, oxodibenzocycloheptenyl, dihydrodibenzocycloheptenyl, oxodihydrodibenzocycloheptenyl, dihydrodibenzocyclooctenyl, tetrahydrodibenzocyclooctenyl and oxotetrahydrodibenzocyclooctenyl, bound directly or over a methylene group,
and
the group consisting of furyl, thienyl, pyrrolyl, oxazolyl, isoxazolyl, thiazolyl, isothiazolyl, pyrazolyl, imidazolyl, oxadiazolyl, thiadiazolyl, triazolyl, pyridyl, pyrazinyl, pyridazinyl, pyrimidinyl, triazinyl, imidazothiazolyl, benzofuryl, dihydrobenzofuryl, benzothienyl, dihydrobenzothienyl, indolyl, indolinyl, oxoindolinyl, dioxoindolinyl, benzoxazolyl, oxobenzoxazolinyl, benzisoxazolyl, oxobenzisoxazolinyl, benzothiazolyl, oxobenzthiazolinyl, benzoisothiazolyl, oxobenzoisothiazolinyl, benzimidazolyl, oxobenzimidazolinyl, indazolyl, oxoindazolinyl, benzofurazanyl, benzothiadiazolyl, benzotriazolyl, oxazolopyridyl, oxodihydrooxazolopyridyl, thiazolopyridyl, oxodihydrothiazolopyridyl, isothiazolopyridyl, imidazopyridyl, oxodihydroimidazopyridyl, pyrazolopyridyl, oxodihydropyrazolopyridyl, thienopyrimidinyl, chromanyl, chromanonyl, benzopyranyl, chromonyl, quinolyl, isoquinolyl, dihydroquinolyl, oxodihydroquinolinyl, tetrahydroquinolyl, oxotetrahydroquinolinyl, benzodioxanyl, quinoxalinyl, quinazolinyl, naphthyridinyl, carbazolyl, tetrahydrocarbazolyl, oxotetrahydrocarbazolyl, pyridoindolyl, acridinyl, oxodihydroacridinyl, phenothiazinyl, dihydrodibenzoxepinyl, oxodihydrodibenzoxepinyl, benzocycloheptathienyl, oxobenzocycloheptathienyl, dihydrothienobenzothiepinyl, oxodihydrothienobenzothiepinyl, dihydrodibenzothiepinyl, oxodihydrodibenzothiepinyl, octahydrodibenzothiepinyl, dihydrodibenzazepinyl, oxodihydrodibenzazepinyl, octahydrodibenzazepinyl, benzocycloheptapyridyl, oxobenzocycloheptapyridyl, dihydropyridobenzodiazepinyl, dihydrodibenzoxazepinyl, dihydropyridobenzoxepinyl, dihydropyridobenzoxazepinyl, oxodihydropyridobenzoxazepinyl, dihydrodibenzothiazepinyl, oxodihydrodibenzothiazepinyl, dihydropyridobenzothiazepinyl, and oxodihydropyridobenzothiazepinyl, bound directly or over a methylene group,
R¹³ has the same meaning as R¹², but is selected independently therefrom,
R¹⁴ is selected from hydrogen, hydroxy, methyl, benzyl, phenyl, and,
the group consisting of indanyl, indenyl, naphthyl, dihydronaphthyl, tetrahydronaphthyl, furyl, thienyl, pyrrolyl, oxazolyl, isoxazolyl, thiazolyl, isothiazolyl, pyrazolyl, imidazolyl, oxadiazolyl, thiadiazolyl, triazolyl, pyridyl, pyrazinyl, pyridazinyl, pyrimidinyl, triazinyl, benzofuryl, benzothienyl, indolyl, indolinyl, benzoxazolyl, benzothiazolyl, benzimidazolyl, chromanyl, quinolyl, and tetrahydroquinolyl, bound directly or over a methylene group,
G2 is selected from the residue 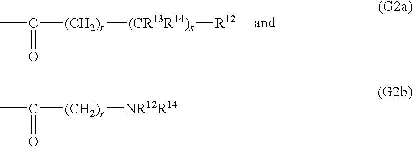
wherein the substituents R¹² and R¹⁴ have the above meanings, or the group
—NR¹²R¹⁴
is a nitrogen-containing heterocycle bound over the nitrogen atom, the nitrogen-containing heterocycle being selected from the group consisting of azetidine, pyrrolidine, piperidine, (1H)tetrahydropyridine, hexahydroazepine, (1H)tetrahydroazepine, octahydroazocine, pyrazolidine, piperazine, hexahydrodiazepine, morpholine, hexahydrooxazepine, thiomorpholine, thiomorpholine-1,1-dioxide, 5-aza-bicyclo[2.1.1]hexane, 2-aza-bicyclo[2.2.1]heptane, 7-aza-bicyclo[2.2.1]heptane, 2,5-diaza-bicyclo[2.2.1]-heptane, 2-aza-bicyclo[2.2.2]octane, 8-aza-bicyclo[3.2.1]octane, 2,5-diazabicyclo[2.2.2]octane, 9-azabicyclo[3.3.1]nonane, indoline, isoindoline, (1H)-dihydroquinoline, (1H)-tetrahydroquinoline, (2H)-tetrahydroisoquinoline, (1H)-tetrahydroquinoxaline, (4H)-dihydrobenzoxazine, (4H)-dihydrobenzothiazine, (1H)-tetrahydrobenzo[b]azepine, (1H)-tetrahydrobenzo[c]azepine, (1H)-tetrahydrobenzo[d]azepine, (5H)-tetrahydrobenzo[b]oxazepine, (5H)-tet-rahydrobenzo[b]thiazepine, 1,2,3,4-tetrahydro-9H-pyrido[3,4-b]indole, (10H)-dihydroacridine, 1,2,3,4-tetrahydroacridanone, (10H)-phenoxazine, (10H)-phenothiazine, (5H)-dibenzazepine, (5H)-dihydrodibenzazepine, (5H)-octahydrodibenzazepine, (5H)-dihydrodibenzodiazepine, (11H)-dihydrodibenzo[b,e]oxazepine, (11H)-dihydrodibenzo[b,e]thiazepine, (10H)-dihydrodibenzo[b,f]oxazepine, (10H)-dihydrodibenzo[b,f]thiazepine, and (5H)-tetrahydrodibenzazocine,
G3 is the residue
—SO₂—(CH₂)ᵣR¹² (G3),
G4 is the residue 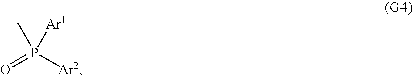
wherein
Ar¹ and Ar² are selected independently of each other from phenyl, pyridyl, and naphthyl,
G5 is the residue
—COR¹⁵ (G5)
wherein
R¹⁵ is selected from trifluoromethyl, C₁-C₆-alkoxy, C₃-C₆-alkenyloxy, and benzyloxy,
wherein aromatic ring systems optionally are substituted independently of each other by one to three substituents independently selected from the group consisting of halogen, cyano, C₁-C₆-alkyl, trifluoromethyl, C₃-C₈-cycloalkyl, phenyl, benzyl, hydroxy, C₁-C₆-alkoxy, C₁-C₆-alkoxy entirely or partially substituted by fluorine; benzyloxy,
phenoxy, mercapto, C₁-C₆-alkylthio, carboxy, C₁-C₆-alkoxycarbonyl, benzyloxycarbonyl, nitro, amino, mono-C₁-C₆-alkylamino, and di-(C₁-C₆-alkyl)-amino,
wherein two adjacent groups in the ring or ring system optionally form an additional ring over a methylenedioxy bridge.

5. Method according to claim 3, wherein:
R¹ is selected from hydrogen, halogen, cyano, methyl, trifluoromethyl, hydroxy, methoxy and methoxycarbonyl,
R² is hydrogen or halogen,
R³ is hydrogen,
R⁴ is selected from hydrogen, C₁-C₃-alkyl and hydroxy,
k is 0 or 1,
A is selected from C₂-C₆-alkenylene, optionally substituted once or twice by hydroxy or fluorine, or C₄-C₆-alkadienylene, optionally substituted by one or two fluorine atoms, and 1,3,5-hexatrienylene
D is selected from C₂-C₈-alkylene, optionally substituted by methyl or hydroxy C₂-C₈-alkenylene, optionally substituted by methyl or hydroxy, wherein the double bond optionally is to ring E, and
the group consisting of C₂-C₈-alkylene and C₂-C₈-alkenylene, wherein one to three methylene units are isosterically replaced by O, NH, N(CH₃), N(COCH₃), N(SO₂CH₃) or CO,
E is selected from the residues 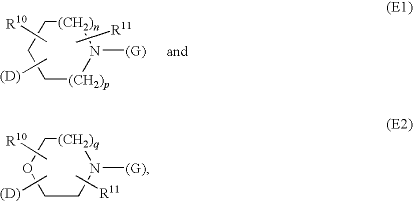
wherein the heterocyclic ring optionally has a double bond and
n and p are, independent of each other, 0, 1, 2 or 3, with the proviso that n+p≦3
q is 1 or 2,
R¹⁰ is selected from hydrogen, methyl and hydroxyl,
R¹¹ is hydrogen or an oxo group adjacent to the nitrogen atom,
G is selected from hydrogen, C₃-C₈-cycloalkyl, methoxycarbonyl, tert-butoxycarbonyl, benzyloxycarbonyl, trifluoroacetyl, diphenylphosphinoyl and the residues
—(CH₂)ᵣ—(CR¹³R¹⁴)ₛR¹² (G1) 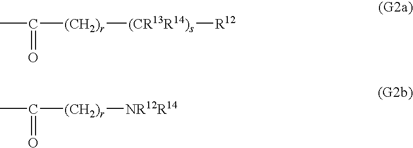
and
—SO₂—(CH₂)ᵣR¹² (G3)
wherein
r is 0, 1 or 2,
s is 0 or 1,
R¹² is selected from hydrogen, methyl, benzyl, phenyl.
the group consisting of indanyl, indenyl, oxoindanyl, naphthyl, dihydronaphthyl, tetrahydronaphthyl, oxotetrahydronaphthyl, flourenyl, oxofluorenyl, anthryl, dihydroanthryl, oxodihydroanthryl, dioxodihydroanthryl, dibenzocycloheptenyl, and oxodibenzocycloheptenyl, dihydrodibenzocycloheptenyl, oxodihydrodibenzocycloheptenyl bound directly or over a methylene group, and
the group consisting of furyl, thienyl, pyrrolyl, oxazolyl, isoxazolyl, thiazolyl, isothiazolyl, pyrazolyl, imidazolyl, oxadiazolyl, thiadiazolyl, triazolyl, pyridyl, pyrazinyl, pyridazinyl, pyrimidinyl, imidazothiazolyl,
benzofuryl, dihydrobenzofuryl, benzothienyl, dihydrobenzothienyl, indolyl, indolinyl, oxoindolinyl, dioxoindolinyl, benzoxazolyl, oxobenzoxazolinyl, benzisoxazolyl, oxobenzisoxazolinyl, benzothiazolyl, oxobenzthiazolinyl, benzoisothiazolyl, oxobenzoisothiazolinyl, benzimidazolyl, oxobenzimidazolinyl, benzofurazanyl, benzothiadiazolyl, benzotriazolyl, oxazolopyridyl, oxodihydrooxazolopyridyl, thiazolopyridyl, oxodihydrothiazolopyridyl, isothiazolopyridyl, imidazopyridyl, oxodihydroimidazopyridyl, pyrazolopyridyl, thienopyrimidinyl, chromanyl, chromanonyl, benzopyranyl, chromonyl, quinolyl, isoquinolyl, dihydroquinolyl, oxodihydroquinolinyl, tetrahydroquinolyl, oxotetrahydroquinolinyl, benzodioxanyl, quinoxalinyl, quinazolinyl, naphthyridinyl, carbazolyl, tetrahydrocarbazolyl, oxotetrahydrocarbazolyl, pyridoindolyl, acridinyl, oxodihydroacridinyl, phenothiazinyl, dihydrodibenzoxepinyl, benzocycloheptathienyl, oxobenzocycloheptathienyl, dihydrothienobenzothiepinyl, oxodihydrothienobenzothiepinyl, dihydrodibenzothiepinyl, oxodihydrodibenzothiepinyl, dihydrodibenzazepinyl, oxodihydrodibenzazepinyl, octahydrodibenzazepinyl, benzocycloheptapyridyl, oxobenzocycloheptapyridyl, dihydropyridobenzoxepinyl, dihydrodibenzothiazepinyl, and oxodihydrodibenzothiazepinyl, bound directly or over a methylene group,
R¹³ is selected from hydrogen, methyl, benzyl and phenyl,
R¹⁴ is selected from hydrogen, hydroxy, methyl, benzyl, phenyl, and
the group consisting of naphthyl, furyl, thienyl, oxazolyl, thiazolyl, pyrazolyl, imidazolyl, oxadiazolyl, thiadiazolyl, pyridyl, benzofuryl, benzothienyl, indolyl, indolinyl, benzoxazolyl, benzothiazolyl, benzim-idazolyl, chromanyl, quinolyl and tetrahydroquinolyl, bound directly or over a methylene group, wherein in formula 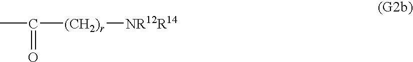
—NR¹²R¹⁴ optionally is selected from pyrrolidine, piperidine, (1H)-tetrahydropyridine, hexahydroazepine, octahydroazocine, piperazine, hexahydrodiazepine, morpholine, hexahydrooxazepine, 2-azabicyclo[2.2.1]heptane, 7-azabicyclo[2.2.1]heptane, 2,5-diazabicyclo[2.2.1]heptane, 8-azabicyclo[3.2.1]octane, 2,5-diazabicyclo[2.2.2]octane, indoline, isoindoline, (1H)-dihydroquinoline, (1H)-tetrahydroquinoline, (2H)-tetrahydroisoquinoline, (1H)-tetrahydroquinoxaline, (4H)-dihydrobenzoxazine, (4H)-dihydrobenzothiazine, (1H)-tetrahydrobenzo[b]azepine, (1H)-tetrahydrobenzo[d]azepine, (5H)-tetrahydrobenzo[b]oxazepine, (5H)-tetrahydrobenzo[b]thiazepine, 1,2,3,4-tetrahydro-9H-pyrido[3,4-b]indol, (10H)-dihydroacridine, 1,2,3,4-tetrahydroacridanone, (5H)-dihydrodibenzazepine, (5H)-dihydrodibenzodiazepine, (11H)-dihydrodibenzo[b,e]oxazepine, (11H)-dihydrodibenzo[b,e]thiazepine, (10H)-dihydrodibenzo[b,f]oxazepine and (5H)-tetrahydrodibenzazocine

6. Method according to claim 3, wherein:
R¹ is selected from hydrogen, fluorine, chlorine, bromine, methyl, trifluoromethyl and hydroxy,
R² and R³ are hydrogen,
R⁴ is hydrogen or hydroxy,
k is 0 or 1,
A is C₂-C₄-alkenylene, which is optionally substituted by fluorine,
D is selected from C₂-C₆-alkylene, C₂-C₆-alkenylene, wherein the double bond optionally is to ring E, and the group consisting of C₂-C₆-alkylene and C₂-C₆-alkenylene, wherein a methylene unit is isosterically replaced by O, NH, N(CH₃) or CO, or an ethylene group is isosterically replaced by NH—CO
or CO—NH, or a propylene group is isosterically replaced by NH—CO—O or O—CO—NH,
E is selected from pyrrolidine, piperidine, 1,2,5,6-tetrahydropyridine, hexahydroazepine, morpholine and hexahydro-1,4-oxazepine, wherein the heterocyclic ring optionally is substituted by an oxo group adjacent to the nitrogen atom,
G is selected from hydrogen, tert-butoxycarbonyl, diphenylphosphinoyl, and one of the residues
—(CH₂)ᵣ—(CR¹³R¹⁴)_{S}—R¹² (G1) 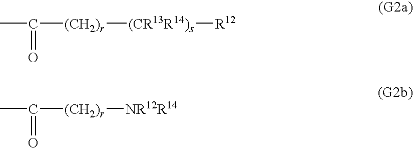
and
—SO₂—(CH₂)ᵣR¹² (G3)
wherein
r is 0 or 1,
s is 0 or 1,
R¹² is selected from hydrogen, methyl, benzyl, phenyl.
the group consisting of indenyl, oxoindanyl, naphthyl, tetrahydronaphthyl, flourenyl, oxofluorenyl, anthryl, dihydroanthryl, oxodihydroanthryl, dioxodihydroanthryl and dibenzocycloheptenyl, dihydrodibenzocycloheptenyl, bound directly or over a methylene group, and
the group consisting of furyl, thienyl, oxazolyl, thiazolyl, imidazolyl, oxadiazolyl, thiadiazolyl, pyridyl, pyrazinyl, pyrimidinyl, imidazothiazolyl,
benzofuryl, benzothienyl, indolyl, oxoindolinyl, dioxoindolinyl, benzoxazolyl, oxobenzoxazolinyl, benzothiazolyl, oxobenzthiazolinyl, benzimidazolyl, oxobenzimidazolinyl, benzofurazanyl, benzotriazolyl, oxazolopyridyl, oxodihydrooxazolopyridyl, thiazolopyridyl, oxodihydrothiazolopyridyl, chromanyl, chromanonyl, benzopyranyl, chromonyl, quinolyl, isoquinolyl, oxodihydroquinolinyl, tetrahydroquinolyl, oxotetrahydroquinolinyl, benzodioxanyl, quinazolinyl, acridinyl, oxodihydroacridinyl, phenothiazinyl, dihydrodibenzoxepinyl, benzocycloheptathienyl, dihydrothienobenzothiepinyl, dihydrodibenzothiepinyl, oxodihydrodibenzothiepinyl, dihydrodibenzazepinyl, oxodihydrodibenzazepinyl, octahydrodibenzazepinyl, benzocycloheptapyridyl, oxobenzocycloheptapyridyl, and dihydrodibenzothiazepinyl, bound directly or over a methylene group,
R¹³ is selected from hydrogen, methyl, benzyl and phenyl,
R¹⁴ is selected from hydrogen, hydroxy, methyl, benzyl, phenyl, and
the group consisting of naphthyl, furyl, thienyl, pyridyl, benzofuryl, benzothienyl, indolyl, benzoxazolyl, benzothiazolyl, benzimidazolyl, chromanyl, quinolyl and tetrahydroquinolyl, bound directly or over a methylene group, wherein in the formula 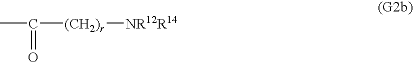
—NR¹²R¹⁴ optionally is selected from pyrrolidine, piperidine, hexahydroazepine, morpholine, 2,5-diazabicyclo[2.2.1]heptane, indoline, isoindoline, (1H)-dihydroquinoline, (1H)-tetrahydroquinoline, (2H)-tetrahydroisoquinoline, (1H)-tetrahydrobenzo[b]azepine, (1H)-tetrahydrobenzo[d]azepine, (5H)-tetrahydrobenzo[b]oxazepine, (5H)-tetrahydrobenzo[b]thiazepine, 1,2,3,4-tetrahydroacridanone, (5H)-dihydrodibenzazepine, (11H)-dihydrodibenzo[b,e]oxazepine, and (11H)-dihydrodibenzo[b,e]thiazepine,
wherein aromatic ring systems optionally are substituted, independently of each other, by one to three substituents which are independently selected from the group consisting of halogen, cyano, C₁-C₆-alkyl, trifluoromethyl, C₃-C₈-cycloalkyl, phenyl, benzyl, hydroxy, C₁-C₆-alkoxy, C₁-C₆-alkoxy which is entirely or partially substituted by fluorine;
benzyloxy, phenoxy, mercapto, C₁-C₆-alkylthio, carboxy, C₁-C₆-alkoxycarbonyl, benzyloxycarbonyl, nitro, amino, mono-C₁-C₆-alkylamino and di-(C₁-C₆-alkyl)-amino, wherein two adjacent groups on the aromatic ring or ring system optionally form an additional ring over a methylenedioxy bridge.

7. Method according to claim 3, wherein:
R¹ is selected from hydrogen, fluorine, methyl, trifluoromethyl and hydroxy,
R² and R³ are hydrogen,
R⁴ is hydrogen or hydroxy,
k is 0,
A is ethenylene or 1,3-butadienylene
D is C₂-C₆-alkylene or C₂-C₆-alkenylene, wherein the double bond optionally is to ring E,
E is selected from pyrrolidine, piperidine, hexahydroazepine and morpholine,
G is selected from benzyl, phenethyl, fluorenylmethyl, anthrylmethyl, diphenylmethyl, fluorenyl, dihydrodibenzocycloheptenyl, furylmethyl, thienylmethyl, thiazolylmethyl, pyridylmethyl, benzothienylmethyl, quinolylmethyl, phenyl-thienylmethyl phenyl-pyridylmethyl, dihydrodibenzoxepinyl, dihydrodibenzothiepinyl,
acetyl, pivaloyl, phenylacetyl, diphenylacetyl, diphenylpropionyl, naphthylacetyl, benzoyl, naphthoyl, anthrylcarbonyl, oxofluorenylcarbonyl, oxodihydroanthrylcarbonyl, dioxodihydroanthrylcarbonyl, furoyl, pyridylcarbonyl, chromonylcarbonyl, quinolylcarbonyl,
naphthylaminocarbonyl, dibenzylaminocarbonyl, benzylphenylaminocarbonyl, diphenylaminocarbonyl, indolinyl-1-carbonyl, dihydrodibenzazepin-N-carbonyl, tetrahydroquinolinyl-N-carbonyl, tetrahydrobenzo[b]azepinyl-N-carbonyl,
methanesulfonyl, phenylsulfonyl, p-toluenesulfonyl, naphthylsulfonyl, quinolinsulfonyl, and diphenylphosphinoyl,
wherein aromatic ring systems optionally are substituted independently of each other by one to three substituents which are independently selected from the group consisting of halogen, cyano, C₁-C₆-alkyl, trifluoromethyl, C₃-C₈-cycloalkyl, phenyl, benzyl, hydroxy, C₁-C₆-alkoxy, C₁-C₆-alkoxy, entirely or partially substituted by fluorine; benzyloxy, phenoxy, mercapto, C₁-C₆-alkylthio, carboxy, C₁-C₆-alkoxycarbonyl, benzyloxycarbonyl, nitro, amino, mono-C₁-C₆-alkylamino and di-(C₁-C₆-alky)-amino, wherein two adjacent groups in the ring or ring system optionally form an additional ring over a methylenedioxy bridge.

8. Method according to claim 1, wherein the inhibitor is a compound selected from
N-[4-(1-methylsulfonylpiperidin-4-yl)-butyl]-3-(pyridin-3-yl)-acrylamide,
N-{4-[1-(2-naphthylsulfonyl)-piperidin-4-yl]-butyl}-3-(pyridin-3-yl)-acrylamide,
N-{4-[1-(2-naphthylsulfonyl)-piperidin-4-yl]-butyl}-5-(pyridin-3-yl)-2,4-pentadienoic acid amide,
N-{4-[1-(1-naphthylaminocarbonyl)-piperidin-4-yl]-butyl}-3-(pyridin-3-yl)-acrylamide,
N-[4-(1-diphenylaminocarbonyl-piperidin-4-yl)-butyl]-3-(pyridin-3-yl)-acrylamide,
N-[4-(1-diphenylaminocarbonyl-piperidin-4-yl)-butyl]-5-(pyridin-3-yl)-2,4-pentadienoic acid amide,
N-{4-[1-(10,11-dihydrodibenzo[b,f]azepin-5-yl-carbonyl)-piperidin-4-yl]-butyl}-3-(pyridin-3-yl)-acrylamide, and
N-[4-(1-diphenylphosphinoyl-piperidin-4-yl)-butyl]-3-(pyridin-3-yl)-acrylamide
or as a pharmaceutically acceptable acid addition salt thereof.

9. Method according to claim 1, wherein the inhibitor is a compound selected from
N-[4-(1-acetylpiperidin-4-yl)-butyl]-3-(pyridin-3-yl)-acrylamide,
N-[4-(1-diphenylacetyl-piperidin-4-yl)-butyl]-3-(pyridin-3-yl)-acrylamide,
N-{4-[1-(3,3-diphenylpropionyl)-piperidin-4-yl]-butyl}-3-(pyridin-3-yl)-acrylamide,
N-[4-(1-benzoylpiperidin-4-yl)-butyl]-3-(pyridin-3-yl)-acrylamide,
N-[4-(1-benzoylpiperidin-4-yl)-butyl]-5-(pyridin-3-yl)-2,4-pentadienoic acid amide, and N-{4-[1-(9-oxo-9H-fluoren-4-yl-carbonyl)-piperidin-4-yl]-butyl}-3-(pyridin-3-yl)-acrylamide, or as a pharmaceutically acceptable acid addition salt thereof.

10. Method according to claim 1, wherein the inhibitor is a compound selected from
N-{4-[1-(phenylpyridin-3-yl-methyl)-piperidin-4-yl]-butyl}-3-(pyridin-3-yl)-acrylamide,
N-{4-[1-(phenylpyridin-4-yl-methyl)-piperidin-4-yl]-butyl}-3-(pyridin-3-yl)-acrylamide,
N-{4-[1-(6,11-dihydrodibenzo[b,e]oxepin-11-yl)-piperidin-4-yl]-butyl}-3-(pyridin-3-yl)-acrylamide and
N-{4-[1-(6,11-dihydrodibenzo[b,e]thiepin-11-yl)-piperidin-4-yl]-butyl}-3-(pyridin-3-yl)-acrylamide,
or as a pharmaceutically acceptable acid addition salt thereof.

11. Method according to claim 1, wherein the inhibitor is a compound selected from
N-[7-(1-diphenylmethylpiperidin-4-yl)-heptyl]-3-(pyridin-3-yl)-acrylamide,
N-[8-(1-diphenylmethylpiperidin-4-yl)-octyl]-3-(pyridin-3-yl)-acrylamide,
N-[3-(1-diphenylmethylpiperidin-4-yloxy)-propyl]-3-(pyridin-3-yl)-acrylamide, and
N-[3-(1-benzylpiperidin-4-yloxy)-propyl]-3-(pyridin-3-yl)-acrylamide
or as a pharmaceutically acceptable acid addition salt thereof.

12. Method according to claim 1, wherein the inhibitor is a compound selected from
N-[2-(1-diphenylmethylpiperidin-4-yl)-ethyl]-5-(pyridin-3-yl)-2,4-pentadienoic acid amide,
N-[4-(1-diphenylmethylpiperidin-4-yl)-butyl]-5-(pyridin-3-yl)-2,4-pentadienoic acid amide,
N-[5-(1-diphenylmethylpiperidin-4-yl)-pentyl]-5-(pyridin-3-yl)-2,4-pentadienoic acid amide and
N-[6-(1-diphenylmethylpiperidin-4-yl)-hexyl]-5-(pyridin-3-yl)-2,4-pentadienoic acid amide
or as a pharmaceutically acceptable acid addition salt thereof.

13. Method according to claim 1, wherein the inhibitor is (E)-N-[4-(1-benzoylpiperidin-4-yl)butyl]-3-(pyridine-3-yl)acrylamide.

14. Method according to claim 1, wherein the inflammatory disease is rheumatoid arthritis.

15. Method according to claim 1, wherein the inflammatory disease is endotoxemia.

16. A pharmaceutical kit comprising at least an effective amount of an inhibitor of the formation of nicotinamide adenyl dinucleotide together with instructions for use in the treatment of inflammatory diseases.

17. A pharmaceutical kit according to claim 16, comprising instructions for use in the treatment of rheumatoid arthritis.

18. A pharmaceutical kit according to claim 16, comprising instructions for use in the treatment of endotexemia.

19.-22. (canceled)
